(19) United States
(12) Patent Application Publication
(10) Pub. No.: US 2012/0095559 A1
(43) Pub. Date: Apr. 19, 2012

(54) INTERVERTEBRAL SPINAL IMPLANT, INSTALLATION DEVICE AND SYSTEM

(76) Inventors: John C. Woods, Port Washington, NY (US); Damon L. Franklin, Queens Village, NY (US)

(21) Appl. No.: 12/906,903

(22) Filed: Oct. 18, 2010

(51) Int. Cl. A61F 2/44

(52) U.S. Cl. 623/17.11

(57) ABSTRACT

Improved interbody spinal implant devices and related instrumentation used for surgical installation of such implant devices for use in spinal fusion surgeries. The spinal implant devices are configured with apertures preferably used in conjunction with the instrumentation of the invention to improve the retention of bone graft material within the implant during installation. The invention also includes improved implants with deployable spike mechanisms.

### FIELD OF INVENTION

The present invention relates to interbody (also termed intervertebral) spinal implant devices and the instrumentation used for surgical installation of such devices and more particularly, to an intervertebral implant and installation tool/device configured for improved sizing, improved installation and maneuverability within interbody disc spaces (also termed resected spaces), improved structural support and stability, and/or improved retention of bone graft material during installation.

### BACKGROUND OF THE INVENTION

The human spine (also referred to as the backbone or vertebral column) is a curved column typically consisting of thirty three vertebrae, the sacrum, intervertebral/spinal discs, and the coccyx. The spine houses and protects the spinal cord in the spinal canal. The vertebrae provide the support and structure of the spine while the discs, located between the vertebrae, act as cushions or “shock absorbers” and also provide some degree of flexibility and motion of the spinal column.

The vertebral column has several curved regions, termed the cervical, thoracic, lumbar, and pelvic regions. The cervical region is the upper most portion of the spine near the neck and consists of vertebrae designated C1-C7. The thoracic region is below the cervical region consisting of vertebrae designated T1-T12. The lumbar region is next continuing down the spine with vertebrae designated L1-L5 in a generally curved shape described as a lordotic curve (“lordosis”). The lumbar region is convex towards the anterior of the body and the convexity of the lower three vertebrae L3-L5 (the degree of lordosis) is typically much greater than that of the upper two vertebrae L1-L2. The sacral region consisting of five fused vertebrae S1-S5 follows next and finally the coccygeal region having four fused vertebrae and a tailbone.

Vertebrae generally increase in size from the top of the spine near the neck to the bottom near the coccyx. The vertebrae generally increase in height, width and depth going down the spine from the neck. The general shape of vertebrae is oval or bean shaped, short in height, typically with slightly concave upper and lower surfaces sometimes with more than one low point (e.g., dimple) on the surface. From one person to the next, the size of vertebrae and the spine varies. Adjacent surfaces of adjacent vertebral bodies in each spine (e.g., the lower (inferior) surface of L2 and the upper (superior) surface of L3) are not usually identical in size or geometry. Typically, the overall area of the inferior surface of L2 is smaller than the overall area of the superior surface of L3.

There are typically 23 discs in the human spine. Six discs in the neck (cervical region), twelve in the middle back (thoracic region), and five in the lower back (lumbar region).

A spinal disc is made up of the nucleus pulposus in the center portion of the disc which, amongst other functions, functions as a ligament and binds the adjacent vertebrae together. The annulus fibrosus surrounds the nucleus pulposus, is more fibrous (tougher) than the nucleus pulposus, and holds the highly pressurized nucleus pulposus in place. The annulus is made of fifteen to twenty five concentric sheets of collagen that are called lamellae (a tough cartilage-like substance) arranged in a special configuration that makes them extremely strong and assists in their job of containing the pressurized nucleus pulposus.

Each disc within an interbody space, such as, for example, the disc within the L2-L3 interbody space, is connected to the respective vertebral endplates (the surface on each adjacent vertebra) through fiberous material. A disc typically occupies the entire interbody space and in some instances may also extend slightly past the outer edges of both vertebral bodies much like a sandwiched or compressed O-ring initially sized the same (the outside dimension) as the two objects between which it is placed and then compressed. As the O-ring compresses, the edges of the O-ring “swell out” past the edges of the objects. Discs, although strong, are also compressible and conform to the spatial dimensions of the interbody space, including the generally concave configuration of most endplates (the endplate on the superior surface of S1 is usually closer to a flat configuration) and discs compress and stretch as needed to allow for loading/unloading and movement of the spine.

One cause of back pain is damaged or diseased discs which affect the structure of the spine, its configuration, the interbody spaces, the surrounding nerves including the spinal nerves within and outside the spinal column, and surrounding muscles. A wide variety of disc deformities, such as tears, cracks, flattening, bulges, ruptures, or herniations affect the function of the spine and may cause back pain. In some instances, osteoporosis, a decrease in bone mass and weakening of the bones, results in compression fractures of vertebra and displacement of discs and vertebrae causing pressure on nerves and/or muscles. Spondylolisthesis is yet another condition where the shifting forward of one or more of the vertebrae causes pressure (a pinching of the nerves. Various treatments for back pain and spinal deformities currently exist including spinal fusion surgery wherein a troubled disc is at least partially removed (a process termed a discectomy), an implant is installed (sometimes with the intention to decompress vertebrae and improve spinal curvature), and bone grows between the two adjacent vertebrae (sometimes through the implant) thereby fusing two vertebrae together with the desired spacing and locations.

The discectomy process is complicated by the surgeon's accessibility to the interbody space and the surgeon's desire to keep a safe distance from nerves, arteries, veins and the spinal cord. This is particularly true for cases with spinal compression wherein the distance between vertebral bodies has lessened from its original/starting distance (and in some instances the vertebral bodies may even be in direct contact with each other) because access to the interbody space limits usage of the instrumentation available for removal of the disc. When it is desirable to decompress the spine (increase the spacing between vertebrae), a ramp device is used to spread and hold the vertebrae apart during the discectomy and also during sizing and installation of the intervertebral implant.

Once the disc is removed and the endplates of the vertebrae are exposed, an intervertebral implant is then sized to fit within the evacuated disc space. The sizing it typically performed using a metal sizing device in about the same configuration as the anticipated final implant device with a long rod/handle attached to the implant. Notably, most intervertebral implants that include ridges, spikes, or serrations on their surfaces to dig/grip into the vertebral endplates for secure placement of the device do not have those parts of the devices on the sizing instrument. The reason for keeping those ridges, spikes, or serrations off the sizing implant(s) is to avoid damage to the endplate(s) caused during the sizing process where the implant is typically impacted into the disc space. This is especially true for compressed vertebrae. The obvious disadvantage with current sizing devices, however, is that they are not the same size as the final implant due to the altered configuration with the ridges, spikes, or serrations. Once the correct implant size is determined the actual intervertebral implant is oftentimes filled with bone graft material and then installed in the interbody space again impacting the implant into the disc space. When bone graft material is used in the implant, it is placed (packed) within recesses of the implant prior to the installation. The bone graft material is intended to promote bone growth between vertebrae. The bone graft material may be autograft, allograft, or other comparable substances that promote the growth of bone between vertebrae, inside the implants, and sometimes around the implant, eventually resulting in fusion of two vertebrae.

The intervertebral implant itself is primarily intended to provide structural stability to the spine in the absence of the disc particularly when weight is loaded/placed on the spine, such as, for example, in a standing position. Movement of the implant after installation is detrimental to the fusion process.

Various surgical methods and approaches are known in the art for performing spinal fusion surgery resulting in installation of an intervertebral implant. For example, it is possible to perform the surgery using an anterior (from the front of the body) approach for the incision and access to the spine, a posterior (from the back) approach, or a lateral (from a side) approach. Each of the aforementioned approaches has its advantages and disadvantages and a surgeon typically has a preferred approach depending upon the facts and circumstances for a specific case.

Anterior interbody fusion procedures generally have the advantages of accessibility to the disc space (usually less obstructions and thus easier endplate preparation and exposure), reduced operative times and reduced blood loss. Further, anterior procedures do not interfere with the posterior anatomic structure of the lumbar spine; the back muscles and the nerves remain generally undisturbed. A larger implant can be implanted with an anterior approach than through a posterior approach. Anterior procedures also eliminate the possibility for scarring within the spinal canal which sometimes occurs from posterior procedures and could result in dural sac tears in revision surgery and other complications.

Posterior interbody fusion procedures generally have the advantage of eliminating disturbance to lateral and anterior muscles and organs and as compared to when pedicle screws and rods are used in conjunction with anterior and lateral procedures, the posterior approach has the added benefit of avoiding multiple incision sites.

Relatively recent technological advances for lateral surgical techniques provide for faster patient recovery due, in part, to smaller incisions, avoided disruption to the posterior and anterior muscles, and potential standalone procedures without need for pedicle screws and rods.

A variety of intervertebral implant systems and implants exist in the market. For example, traditional threaded implants involve cylindrical bodies typically packed with bone graft material surgically placed within pre-tapped holes within the interbody disc space. The pre-tapped holes damage the endplates and the location of the implant is not the preferred position because only a relatively small portion of the vertebral endplate is contacted by these cylindrical implants. Accordingly, these implant bodies will likely contact the softer cancellous bone rather than the stronger cortical bone, or apophyseal rim, of the vertebral endplate. There is also a significant risk for the implant moving during and/or after surgery (sinking or settling into the softer cancellous bone of the vertebral body (termed subsidence).

In contrast, open ring or oval shaped cage implant systems are configured to mimic the generally oval or bean shaped contour of the vertebral body surface. The ring shaped cages are typically sized smaller than the entire cortical rim on the end plates and thus those cages do not contact the entire rim. Further, due to the flat upper and lower surfaces of most of these cages, they do not maximize the amount of surface contact with the end plate within the cortical rim. This is one of the many important downsides to most current implants that the present invention helps to address. It is well known in the industry that better fusion rates occur when bone is in compression because bone responds to stress (Wolff s law). Having an implant in contact with more of the surfaces of adjacent endplates should ideally improve fusion and overall success for implant procedures. Some ring or oval implants currently available include a center support down the middle of the implant to improve structural stability but those implants fail to increase the heights of those center support(s) and thus do not conform to the generally concave endplate configurations resulting in poor surface contact between the implant and the endplates.

The ring or oval shaped implants may be made from polyetherether-ketone (PEEK), carbon fiber, titanium or they may be comprised of allograft bone material. PEEK and carbon fiber materials provide for radiolucent cages that provide for better post-op visualization of the healing bone.

At least from a mechanical and structural standpoint, the preferred shape and configuration of the interbody implant is one that conforms to the geometry of the endplates (both endplates forming the intervertebral disc space) and contacts as much as possible of the vertebral body endplates, including the cortical rims.

When performing a spinal fusion surgery, preferably, the endplate (subchondral bone) is roughened to make it bleed but not damaged to the point of breaking through the endplate thereby exposing the softer (cancellous bone). It is also desired and preferred to minimize the damage to the cortical rim particularly when sizing of the implant and during implant installation. Since the outer bone surface of the endplate, the subchondral bone, is stronger than the underlying bone, the cancellous bone, when performing a fusion surgery, it is desirable to minimize the damage and removal of the subchondral bone.

An ideal interbody implant would generally mimic the shape and contour of the disc space meaning it would generally conform to the contours of both endplates contacting the endplates on as much implant surface as possible and providing for the proper degree of lordosis (where spinal curvature exists or is desired). In addition, although vertebral endplates are typically concave, particularly in the lumbar region (except perhaps the endplate on the upper side of S1), most current interbody implants are configured with generally planar/flat upper and lower surfaces (for those with the ridges or serration this refers to the upper most parts of the ridges or serrations and the lower parts as well) resulting in less desirable surface area contact between the implant and the vertebral bodies and a greater chance for post-installation/post-op movement and subsidence.

Often the size and configuration of the intervertebral implant is dictated by the surgical approach for various reasons that include the degree of disc removal achieved, accessibility to the interbody disc space due to surrounding tendons, muscles, arteries, nerve, organs, and bones, accessibility with instrumentation, as well as spacial constraints in the disc space for the implant.

When performing an anterior spinal fusion, the incision is typically larger than when performing other approaches and there is better accessibility to the interbody space for the discectomy and for insertion of the implant. Consequently, for anterior approaches, the implant configurations are typically wider from side to side than they are in length (from front to back).

Posterior approaches provide less accessibility to the interbody space due to the spinal column and surrounding nerves. Consequently, posterior implants are typically narrower than anterior implants. For example cylindrical cage implants, or bean shaped implants that are intended to be inserted and then curved into the interbody space are typically used. The posterior implant is typically not as wide from side to side (as seen when installed) than the anterior implants. The space within which the posterior implant can be inserted into the interbody space is constrained to at least half of the space provided by an anterior approach due to the spinal column.

Lateral approach implants exist that are configured generally narrow as measured along the anterior-posterior axis and relatively long in the side-to-side axis (as viewed when installed), a configuration generally adapted to the surgical approach and accessibility of the interbody space. Lateral implants are also typically smaller and narrower than anterior implants because they need to be placed down a retractor and because the anterior longitudinal ligament remains intact reducing the amount of distraction.

Some of the challenges and disadvantages to current interbody implants and associated installation devices are:
a) that the implants are difficult to install, particularly when a separate ramp device or retractor is needed to separate, distract, and/or decompress vertebrae. Typically, secondary instrumentation is used to keep the disc space distracted during implantation. The use of such instrumentation means that the exposure needs to be large enough to accommodate the instrumentation. If there is a restriction on the exposure size, then the maximum size of the implant available for use is correspondingly limited. The need for secondary instrumentation for distraction during implantation also adds an additional step or two in surgery.
b) the implants, whether with or without spikes, serrations or ridges, damage the subchondral bone of the vertebrae, including the cortical rim, when they are forced between vertebrae during installation, an especially undesired result for osteoporotic bone;
c) the implants are not configured to maximize surface contact with the endplates;
d) the implants are not configured to the general convex contour of endplates resulting in poor surface contact between the implant and the endplates which decreases stability of the implant, reduces structural integrity and increases the chances for subsidence;
e) the sizing procedure is complicated by the fact that the sizing instruments are as difficult to insert and remove as the actual implants themselves; with and for those implants containing grippers, ridges, or spikes on the upper and/or lower surfaces of the implant the sizing instrument does not include the grippers, ridges, or spikes resulting in a sizing device that is not the exact same size as the actual implant;
f) the bone graft material has a tendency to fall out of the implant during installation and/or sometimes when the implant's positioning is adjusted within the interbody disc space, particularly when the implant is partially removed from the disc space; movement during installation of the final implant increases the chance that graft material used within the implant will move, possibly fall out if the implants is removed from the disc space in whole or in part, which requires additional labor to repack the implant, a difficult and time consuming task especially when complete removal and reinstallation of the implant is necessary;
g) implants containing grippers or ridges or predisposed spikes often cause damage to the subchondral bone on the vertebrae, particularly on the cortical rims and the sides of the vertebrae when they are forced into the interbody disc space;
h) implants containing grippers or ridges or predisposed spikes do not go in smoothly which creates greater chance for movement or displacement of the graft material used within the implant and complete removal and repacking of the implant is a difficult and time consuming task;
i) for those implants with deployable spikes into the endplate(s) to hold the implant in place, it is necessary to strike a pin or rod in order to generate enough force to deploy the spike(s) into the bone which could move the already positioned implant and once deployed, the spikes are not retractable;
j) implants are configured with threaded holes that receive threaded insert tools (e.g., a rod) used for installing the device within the interbody space and the threads in the actual implant, which are typically made from PEEK or carbon fiber material, are known to have the threads break or strip during impaction causing difficulty with the installation; and
k) traditional implants are either threaded into place, or have spikes which are designed to prevent expulsion but few exist that are designed to be smooth upon installation thereby allowing for maneuverability within the interbody space and also provide for deployable “spikes” once the desired location is identified.

Accordingly, there is a need for an improved intervertebral spinal implant and an improved installation device that overcomes these and other drawbacks. There is a need for an improved intervertebral spinal implant configured with improved characteristics (structural and mechanical) and/or with an improved installation device or assembly to help retain bone graft material within the implant.

### SUMMARY OF THE INVENTION

The present invention overcomes the foregoing and other shortcomings and drawbacks associated with intervertebral spinal implant devices and installation equipment heretofore known. While the invention will be described in connection with certain embodiments, it will be understood that the invention is not limited to those embodiments. To the contrary, the invention includes all alternatives, modifications and equivalents as may be included within the spirit and scope of the present invention.

The present invention is an improved intervertebral implant for spinal fusions (anterior, lateral and posterior installation) having a ramped front end and a flat back end, a first side and a second side, an upper surface and lower surface and a generally hollow interior.

In accordance with the present invention the ramped front end of the implant assists with separation of two vertebrae during insertion of the implant device. Accordingly, the ramped configuration also improves sizing procedures and the ease with installation of the implant, particularly for compressed vertebrae. The ramped implant configuration also decreases damage to the vertebral endplates and cortical rims during impaction for installation of the implant device in the disc space.

In one embodiment of the present invention, the back end of the implant is configured flat and/or with a flat area(s) to receive the impaction forces (e.g., from a hammer or mallet) frequently used to drive or push the device into an interbody disc space (thereby decompressing the vertebral bodies). Preferably, the impaction forces are transmitted to the implant through an installation device comprising a clamping mechanism and companion contact surface(s) that are configured generally flat to contact the flat area(s) on the implant.

In another embodiment of the invention, at least one of the outside surfaces of the first side and the second side of the implant, when viewed from the top of the implant, are configured to be convex. Accordingly, both the outer surfaces of the first side and the second side of the implant may be convex.

In another embodiment of the invention, at least one of the inside surfaces of the first side and the second side of the implant, when viewed from the top of the implant, are configured to be straight.

In yet another embodiment of the invention, at least one of the upper and lower surfaces of the first side and the second side of the implant, when viewed from the side of the implant, are configured to be convex.

Another embodiment of the present invention is an intervertebral implant for spinal fusions, preferably in the lumbar region, having a ramped front end and a flat back end, a generally convex first side and a generally convex second side, an interior support between the front end and the back end, a generally convex (in at least one direction) upper surface and a generally convex (in at least one direction) lower surface, and two apertures between the a generally convex lower surface and the generally convex upper surface separated by the interior support. The ramp on the front end of the implant device helps separate and/or decompress the vertebrae during installation of the device and in combination with the generally smooth convex shaped lower surface and upper surface, helps to minimize the damage to the end plates, cortical rims and vertebrae. The convexity of the upper and lower surfaces also provides for improved maneuverability of the interbody implant within the interbody space prior to final positioning. The convexity of the upper and lower surfaces also provides an improved ability to remove the interbody implant from the interbody space, if desired, with minimal damage to the vertebrae endplates. The convexity of the upper and lower surfaces generally conforms to the concave geometry of the endplate configurations providing improved structural stability and support in the interbody disc space.

Bone graft material may be packed within the aperture(s) of the implant in accordance with the present invention to promote bone growth and vertebrae fusion. Furthermore, the present invention is also a compressed and/or shaped bone graft material configured to cover the upper and/or lower surface of the implant between the first side and second side and between the front end and the back end. Alternatively, the compressed and/or shaped bone graft material can be configured to cover the upper and/or lower surface of either one of the apertures within the implant. The compressed and/or shaped bone graft material functions to maintain, in place (within the apertures) during and post installation (if the compressed bone graft covers remain in place after insertion into the disc space), the uncompressed/loose bone graft material packed into the recess of the implant—material that sometimes falls out of current implants during installation.

The present invention also includes an installation device used to install the intervertebral implant. The installation device is configured to clamp onto at least the back end of the implant but could also be configured to clamp on the front end or clamp onto both the front end and the back end. In one embodiment, the installation device clamps over the aperture(s) in the implants thereby retaining the bone graft material inside the implant until the clamp is removed. In another embodiment, the installation device is capable of removably clamping to the implant with independent sliding covers on top and/or on bottom of the implant (covering the aperture(s) with the bone graft material) that can be used at the option of the surgeon. The covers help keep the bone graft material in the implant during installation. The installation device can also be used as an impactor and it also includes an opening/cannula down the center axis of the device for insertion of a screw driver that can be used to deploy and retract a deployable spike mechanism in the implant.

The intervertebral implant may also be configured with recesses on the upper and lower portions of the back end and/or front end for improved contact with and attachment to an installation device. The recesses also provide for open areas between the endplates and the intervertebral implant through which the installation device can be removed with minimal disruption to the implant's positioning and the packed bone graft material.

When bone graft material is placed within the aperture(s) of the intervertebral implant the installation device may be used to help maintain the graft material within the aperature(s) of the implant device during installation which decreases the chance for needing to remove and re-pack the implant.

Another embodiment of the invention is an intervertebral implant for spinal fusions having a ramped front end and a flat back end, a generally convex first side and a generally convex second side, an interior support between the front end and the back end, a generally convex (in at least one direction) upper surface and a generally convex (in at least one direction) lower surface, and two apertures between the generally convex lower surface and the generally convex upper surface separated by the interior support, and a deployable spike mechanism located within the interior support. The deployable spike mechanism comprises spikes that are forced into the subchondral bone (and possibly also into the cancellous bone) of at least one of the endplates, preferably both endplates, wherein the spikes deploy from inside the interior support out through the upper surface and/or the lower surfaces. Preferably, the spike mechanism is utilized (the spikes deployed) after the implant is located/positioned within the interbody space. Deployment of the spike mechanism after positioning reduces the damage to the vertebral bodies during insertion and positioning of the implant. The spikes, when deployed, help minimize movement of the implant during additional surgical procedures and post-surgery.

The present invention also includes the improved installation device configured to effectuate the deployment of the spikes while in position in the disc space using a screw and/or impactor.

In one embodiment of the invention, the deployable spike mechanism comprises a screw, a wedge shaped advancement pin, and spikes. Use of the deployable spike mechanism of the present invention eliminates disruptive impact forces associated with conventional spike deployment devices (e.g., a hammer and pin/wedge mechanism). The present invention utilizes a screw positioned in front of a tapered or wedge shaped shaft that forces the spikes out of the implant as the screw is turned/advanced.

In another embodiment of the present invention, the deployable spike mechanism comprises a screw, an advancement pin with slopes and a slanted ridge thereon, and spikes attached to a lower body having slopes and a slanted groove thereon. According to that embodiment, the spike deployment mechanism is both deployable and retractable.

The above and other objects and advantages of the present invention shall be made apparent from the accompanying drawings and the description thereof.

### DESCRIPTION OF THE DRAWINGS

The accompanying drawings, which are incorporated in and constitute a part of this specification, illustrate embodiments of the invention and, together with the general description of the invention given above and the detailed description of an embodiment given below, serve to explain the principles of the present invention. Similar components of the devices are similarly numbered for simplicity.

FIGS. 1 and 2 are perspective, front, side, top and rear views of one embodiment of a intervertebral implant in accordance with the principles of the present invention having a ramped front end member, a flat back end member, convex first side member and second side members, convex upper and lower surfaces (from front to back), and an aperture.

FIG. 3 shows a clamp removably attached to the implant shown in FIGS. 1 and 2.

FIGS. 4, 5 and 6 are perspective, side and top views of one embodiment of the medical clamp of the present invention configured to removably attach to the implant across the front end member and the back end member of an implant covering the bone graft packed aperture of the implant.

FIGS. 7 and 8 are perspective, front, side, top and rear views of one embodiment of a intervertebral implant in accordance with the principles of the present invention having a ramped front end member, a flat back end member, convex first side and second side members, convex upper and lower surfaces (from front to back), an aperture and recesses on the front end member and the back end member.

FIGS. 9 and 10 are perspective, front, side, top and rear views of one embodiment of a intervertebral implant in accordance with the principles of the present invention having a ramped front end member, a flat back end member, convex first side and second side members, convex upper and lower surfaces (from front to back), an aperture, recesses on the front end member and the back end member, and different maximum heights for the first side member and the second side member for lordosis.

FIGS. 11 and 12 are perspective, front, side, top and rear views of one embodiment of a intervertebral implant in accordance with the principles of the present invention having a ramped front end member, a flat back end member, convex first side member and second side member, an aperture, recesses on the front end member and the back end member, and increasing heights for the first side member and the second side member from the front end member to the back end member for lordosis.

FIGS. 13 and 14 are perspective, front, side, top and rear views of one embodiment of a intervertebral implant in accordance with the principles of the present invention having a ramped front end member, a flat back end member, convex first side member and second side member, a convex center member, two apertures, convex upper and lower surfaces (from front to back and from side to side), and recesses on the front end member and the back end member.

FIGS. 15 and 16 are perspective, front, side, top and rear views of one embodiment of a intervertebral implant in accordance with the principles of the present invention having a ramped front end member, a flat back end member, convex first side and second side members, a convex center member, two apertures, convex upper and lower surfaces (from front to back and from side to side), recesses on the front end member and the back end member, and different maximum heights for the first side member and the second side member for lordosis.

FIGS. 17 and 18 are perspective, front, side, top and rear views of one embodiment of a intervertebral implant in accordance with the principles of the present invention having a ramped front end member, a flat back end member, convex first side and second side members, a convex center member, two apertures, convex upper and lower surfaces (from front to back and from side to side), recesses on the back end member, and increasing heights for the first side member and the second side member from the front end member to the back end member for lordosis.

FIGS. 19-22 show one embodiment of a intervertebral implant in accordance with the principles of the present invention having a ramped front end member, a flat back end member, convex first side member and second side member, a convex center member, two apertures, convex upper and lower surfaces (from front to back and from side to side), recesses on the front end member and the back end member, and a deployable and retractable spike mechanism.

FIGS. 23-27 show one embodiment of a intervertebral implant in accordance with the principles of the present invention having a two piece design with a ramped front end member, a flat back end member, convex first side member and second side member, a convex center member, two apertures, convex upper and lower surfaces (from front to back and from side to side), recesses on the front end member and the back end member, and a deployable and retractable spike mechanism.

FIGS. 28, 29, 30 show several embodiments of the implant of the present invention in various sizes and configurations.

FIGS. 31-38 show an embodiment of the installation device of the present invention. FIG. 36 is an exploded perspective view of an embodiment showing the mechanical parts and inner workings of the device. FIGS. 37-38 are perspective views of the installation device removably secured to an implant of the present invention. The installation device in FIGS. 37 and 38 are for implants without a center member. FIG. 38 shows the implant attached to the installation device with the implant rotated about the tip of the installation device.

FIGS. 39 shows an embodiment of the installation device of the present invention. The installation device in FIG. 39 is configures for an implant with a center member.

FIG. 40 show an embodiment of the implant of the current invention with an attached handle for sizing of the implant in a disc space.

### DETAILED DISCLOSURE

In one embodiment of the interbody implant according to the present invention, the implant is a hollow implant having a generally convex first side member, a generally convex second side member, a ramp shaped front end member and a generally flat back end member. The aperture inside the implant extends through the implant. One or both of the upper surfaces and/or the lower surfaces of the implant formed by the first side member, the second side member, the front end member and the back end member are convex shaped. At least one or both of the upper surface and/or lower surface of implant are convex from front to back. The back member of the implant is configured to removably receive (attach with/to) an instrument or handle/clamp that is used by the medical practitioner to place the implant in the patient.

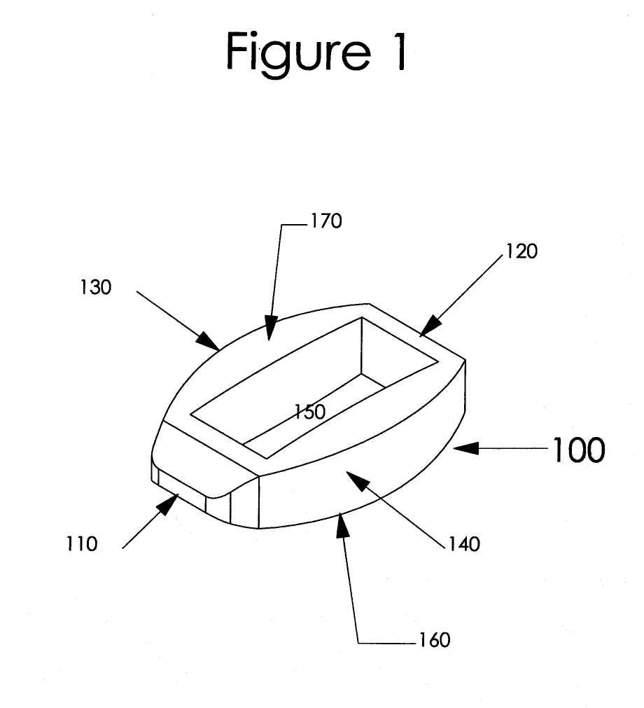

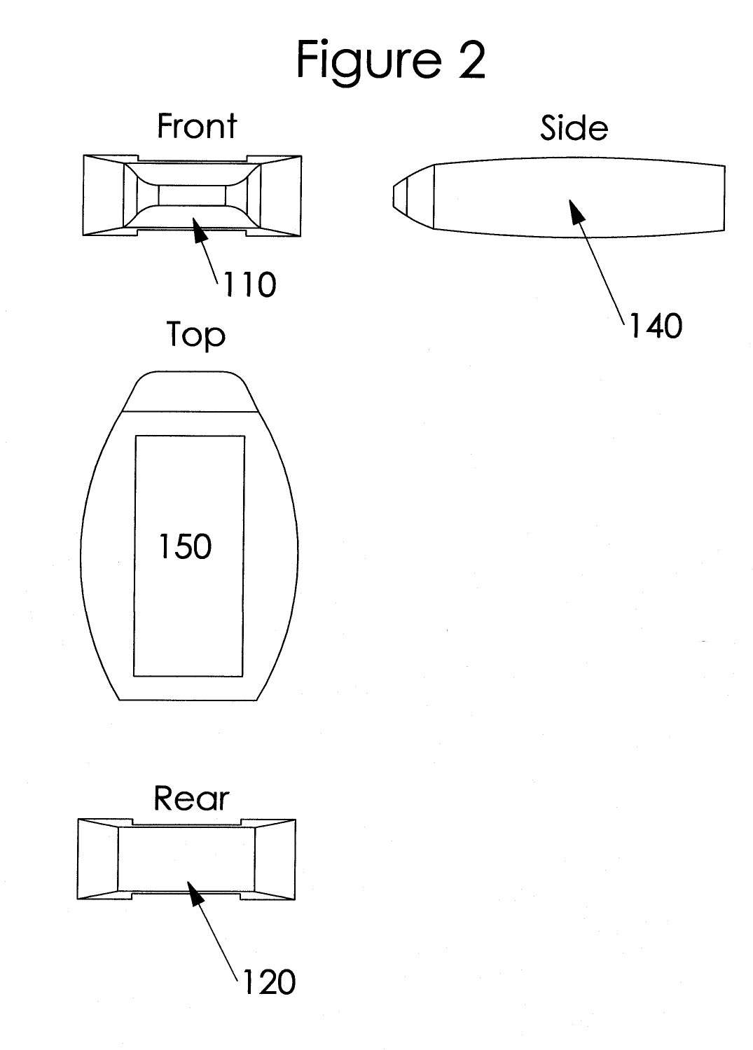

For example, as shown in FIGS. 1-3, implant 100 comprises front end member 110, back end member 120, first side member 130, second side member 140 and aperture 150 generally extending through the implant from the lower surface of the implant (designated 160) to the upper surface of the implant (designated 170). Front end member 110 is ramped upwards towards the back end member 120 which helps minimize damage to the vertebral bodies, including the endplates and cortical rims, during installation of implant 100, especially when the installation requires force/impaction. The upper surface 170 and the lower surface 160 of implant 100 are convex in the direction from the front end member 110 to the back end member 120. It is understood that the invention also includes an implant with only one of the upper surface and the lower surface configured convex, the non-convex surface capable of being flat or concave or irregularly configured. Having convex upper surface 170 and convex lower surface 160 provides for improved insertion ability for implant 100 in a disc space, particularly when separation and/or decompression of the vertebral bodies is necessary. The convex upper surface 170 and convex lower surface 160 also provide for a smooth contact surface with the vertebral bodies which also minimizes damage to the end plates and vertebrae during installation and manipulation of the device in the disc space. The degree of concavity of the upper surface 170 and lower surface 160 is variable and is intended to generally conform to the concave geometry of vertebral endplate configurations providing improved stability and support in the interbody disc space.

The convex outer surface configurations of the first side member 130 and the second side member 140 similarly provide improved conformity with the concave configuration of the vertebral bodies, conform to the general shape of the vertebral bodies, and also provide for improved structural support. A curved wall between two points is longer than a straight wall between the same two points resulting in a greater and more desirable distribution of the same loading placed on the walls. For the embodiment shown in FIGS. 1-3, the convex outer surfaces of first side member 130 and second side member 140 with straight inner surfaces also makes for a thicker wall with greater surface areas and contact surfaces on the upper surface member and lower surface member.

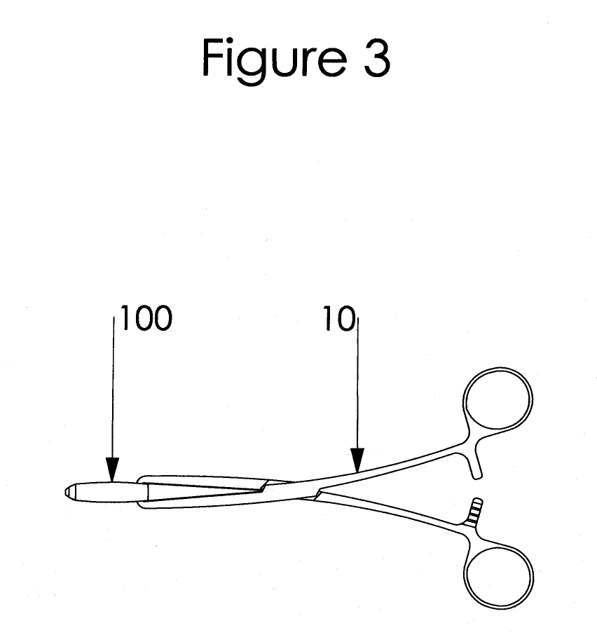

The generally flat outer surface of back end member 120 provides a surface for receipt of impact force applied through an installation device attached to or put against the implant 100 and extending outside the patient to force the implant 100 into the interbody disc space. As shown in FIG. 3, the back end member 120 can be used to removably receive (attach to) an instrument or handle (shown as a clamp 10) that is used by the medical practitioner to hold the implant 100 during placement. Once in place in the disc space, an impactor can be separately applied to/contacted with the outer surface of the back end member 120 to push the implant 100 into the disc space. Holding the implant 100 at the back end member 120 with a clamping device and impacting the outer surface of the back member 120 of an implant is an improvement over existing technologies that utilize threaded holes in the implant and threaded installation devices. For those devices, the threads in the implant sometimes break during impaction.

The present invention also includes an improved medical clamp configured to removably attach to the implant on one or both the front end member and the back end member, e.g., across the implant and the aperture in the implant, preferably in the direction of insertion. The improved clamp of the present invention is configured help keep bone graft material inside the aperture after it is packed and during installation into the disc space. After the implant is in place, the clamp is opened and removed from the implant device leaving the bone graft material in the aperture of the implant within the disc space.

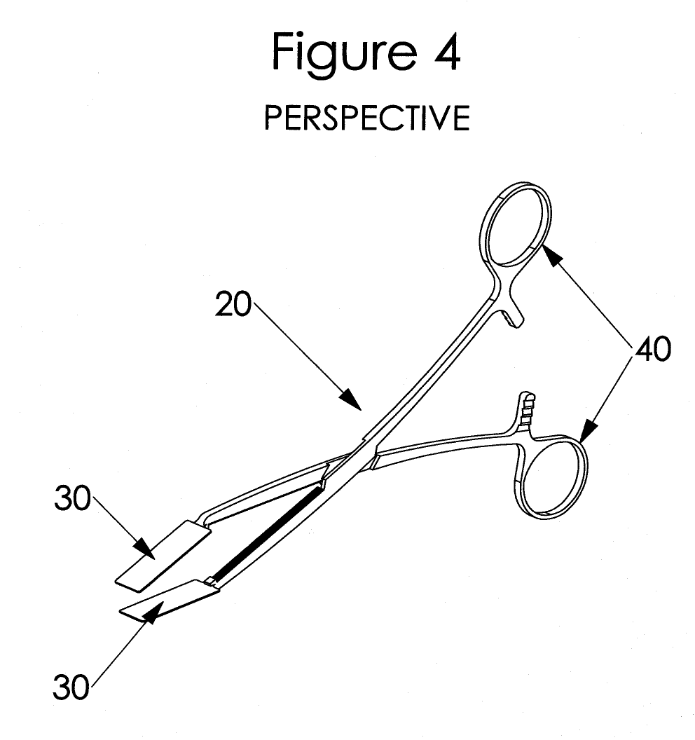

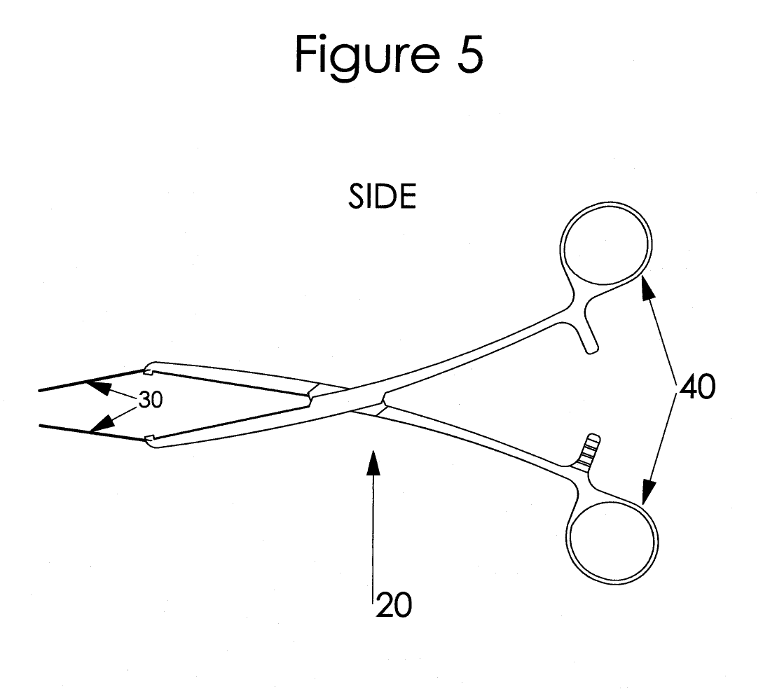

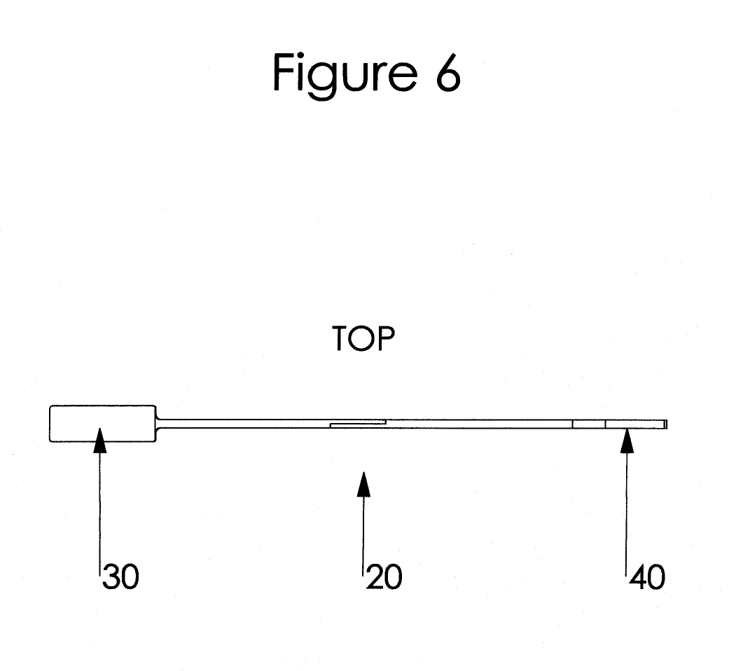

An example clamp is shown in FIGS. 4-6 having elongated clamping portions on the front end of the clamp 20. Clamp 20 includes clamping portions 30 that are used to hold an implant (not shown) across the implant and the aperture in the implant, preferably in the direction of insertion. Once in place in the disc space, the clamping device 20 is opened using handles 40 and clamping device 20 is removed.

In another embodiment, the same clamp is configured with a part of the clamp device 20 in contact with the outer surface of the back end member so that the clamp and implant can be impacted while clamped thereby pushing the implant into the disc space.

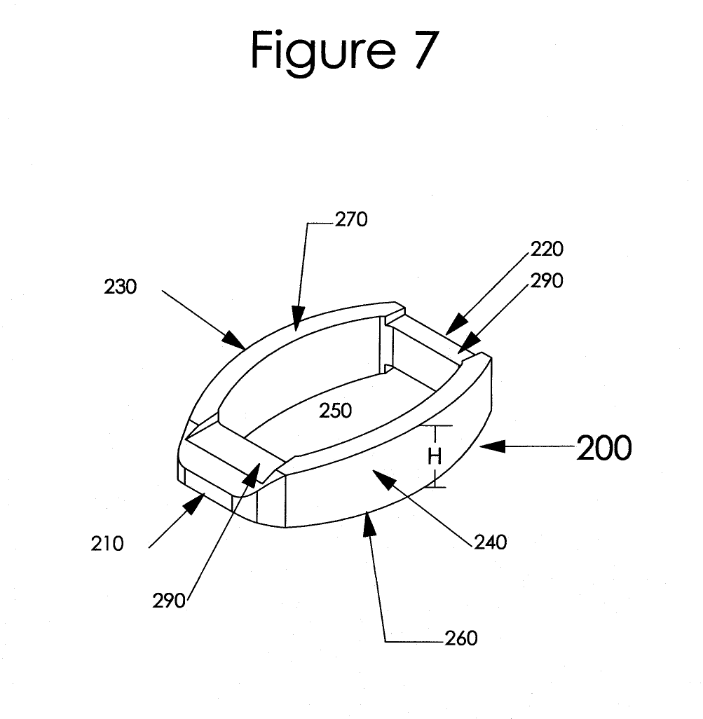

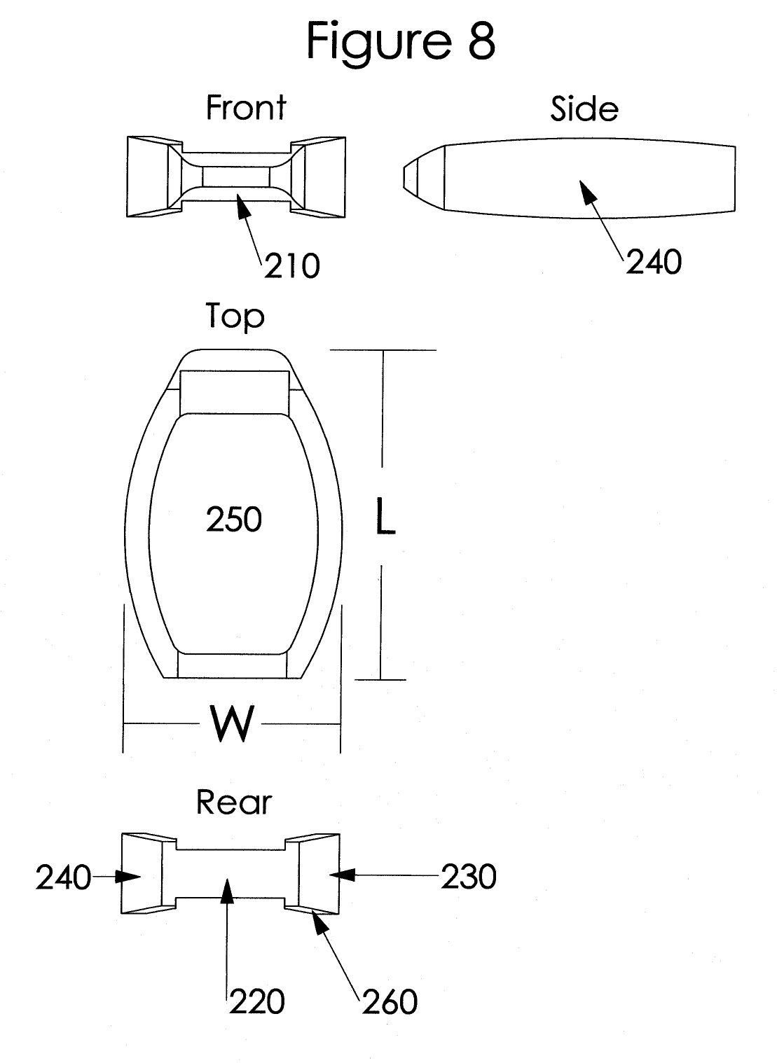

Another embodiment of the implant of the present invention is shown in FIGS. 7-8. Implant 200 comprises front end member 210, back end member 220, first side member 230, second side member 240 and aperture 250 generally extending through the implant from the lower surface of the implant (designated 260) to the upper surface of the implant (designated 270). Front end member 210 is ramped upward towards the back end member 220. The upper surface 270 and the lower surface 260 of implant 200 is convex in the direction from the front end member 210 to the back end member 220. It is understood that the invention also includes an implant with only one of the upper surface and the lower surface configured convex, the non-convex surface capable of being flat, concave or irregular.

The convex configurations of the first side member 230 and the second side member 240 similarly provide improved conformity with the concave configuration of the vertebral bodies and also provide for improved structural support. For the embodiment shown in FIGS. 7-8, the outer surfaces of first side member 230 and the second side member 240 are convex and the inner surfaces are concave. It is understood that the inner surface of the first side member 230 and the second side member 240 may be straight or covex.

Recesses 290 are formed on both of the front end member 210 and the back end member 220 between the first side member 230 and the second side member 240 on both the upper surface 270 and the lower surface 260. Recesses 290 are configured for receiving and removably attaching to/fastening to an installation device (e.g. clamp) used to position and install the implant 200 in the interbody disc space. The size of the recesses 290 may be, but need not be, configured slightly larger than the connecting elements of an installation device for stability between the two. Bone graft material (not shown) may be packed within aperture 250 of implant 200 prior to installation of implant 200 in a disc space. Again, the installation device preferably includes components that cover the aperture 250 at or about at the upper surface 270 and the lower surface 260 of the implant 200 to keep bone graft material in the implant 200.

In the embodiment shown in FIGS. 7 and 8, the configurations of the first side member 230 and the second side member 240, including the lengths, widths, and heights (the height is designated “H”) are equal to each other. It is understood that variations for the dimension(s) for one or both of the side members are possible and included in the scope of the invention. For example, the length (designated “L”), height (designated “H”), and/or width designated “W”) may be increased or decreased to create implants of varying sizes to fit the desired dimensions of the interbody space.

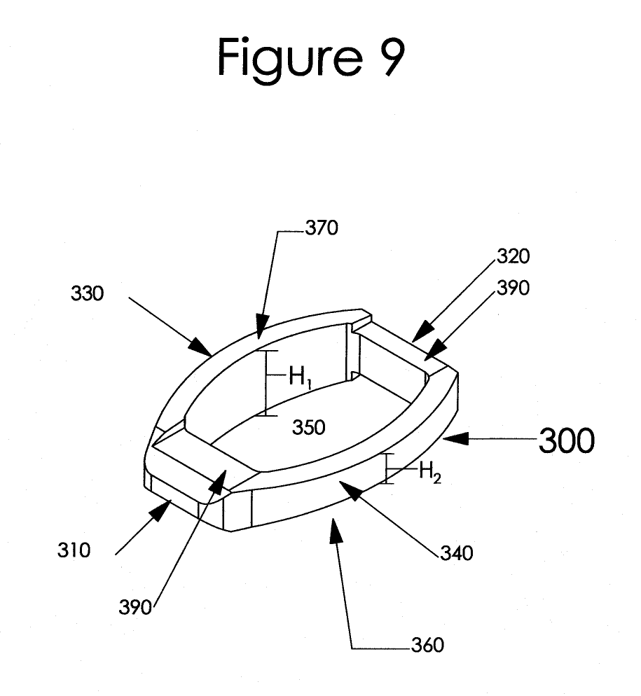

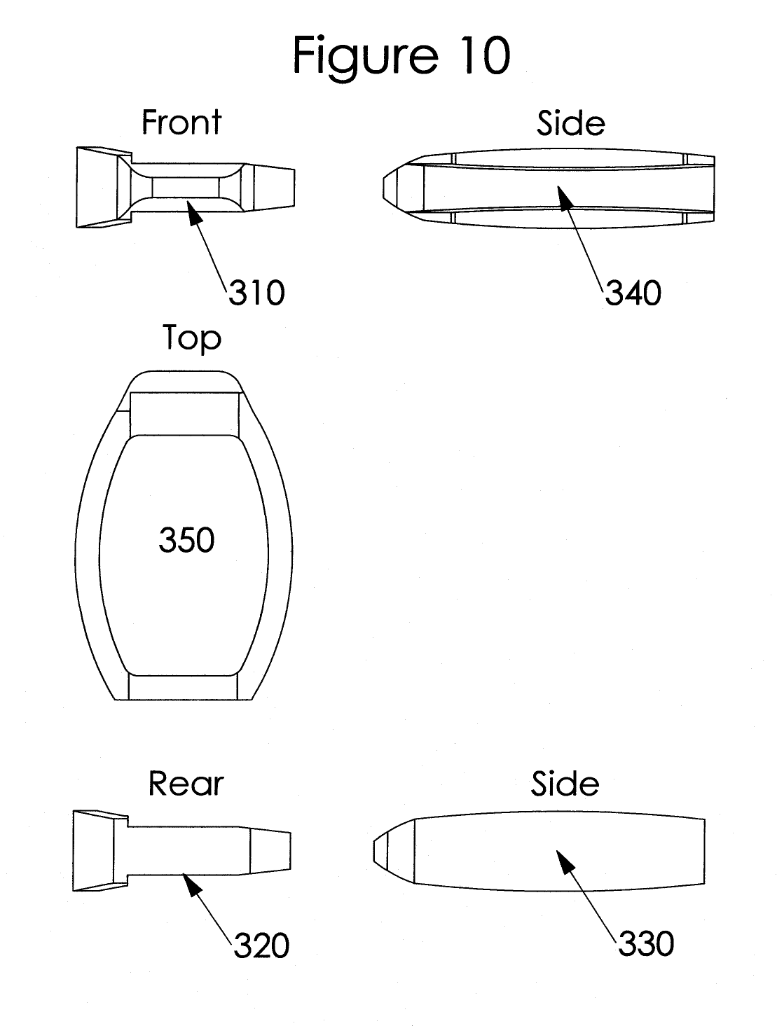

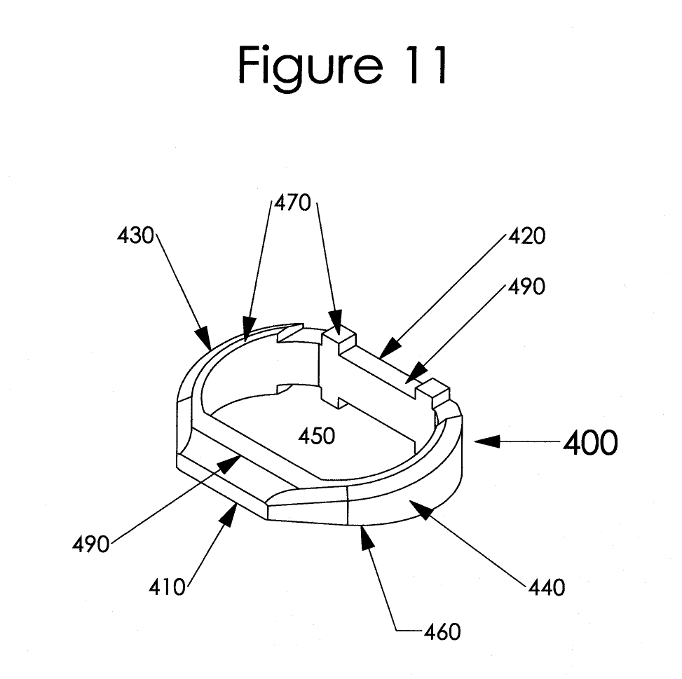

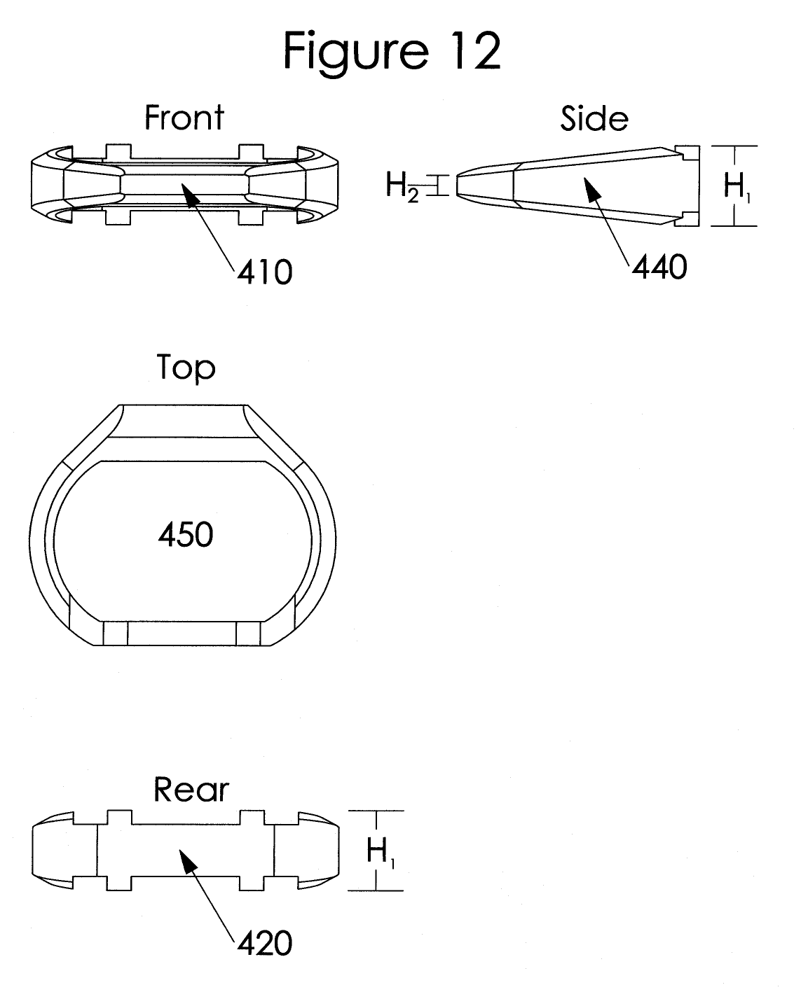

In the embodiment shown in FIGS. 9 and 10, the maximum height H₁ of first side member 330 is larger than the maximum height H₂ of second side member 340. Such a configuration is useful for interbody disc spaces with lordosis or when the intervertebral implant is used to create lordosis between vertebral bodies. This embodiment includes a ramped front end member 310 for a lateral installation. Alternatively, if the present invention for the implant intended for lordosis is configured for an anterior installation, as shown in the embodiment FIGS. 11 and 12, the maximum height H₁ of each the first side 430 and the second side member 440 is positioned in proximity to the back end member 420 which is also the maximum height for the back end member 420. The minimum height H₁ is positioned at the front end member 410.

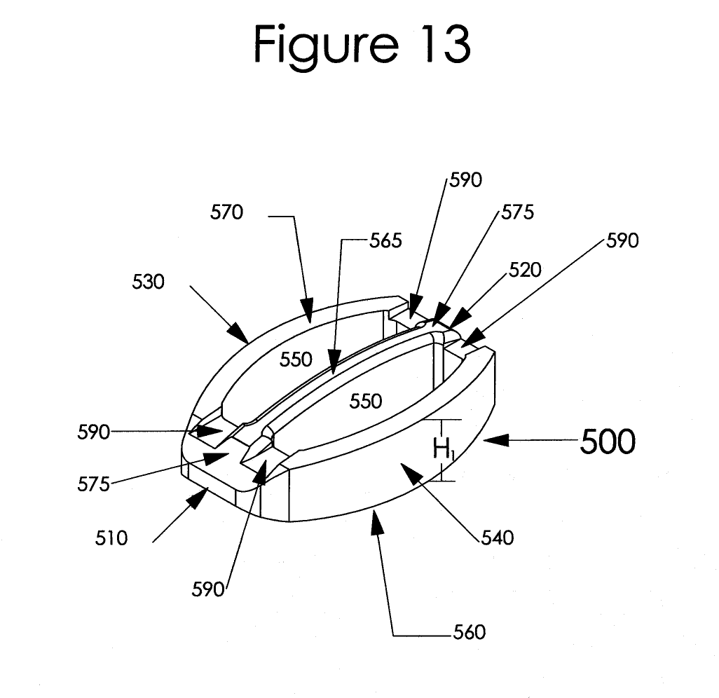

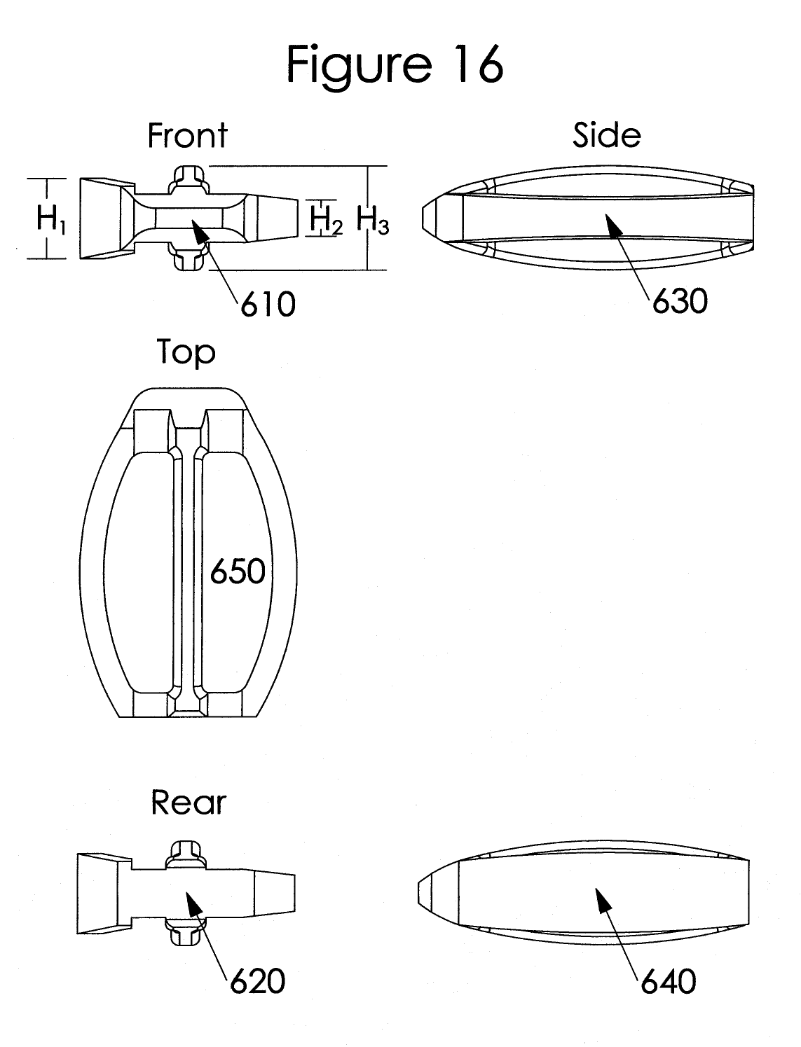

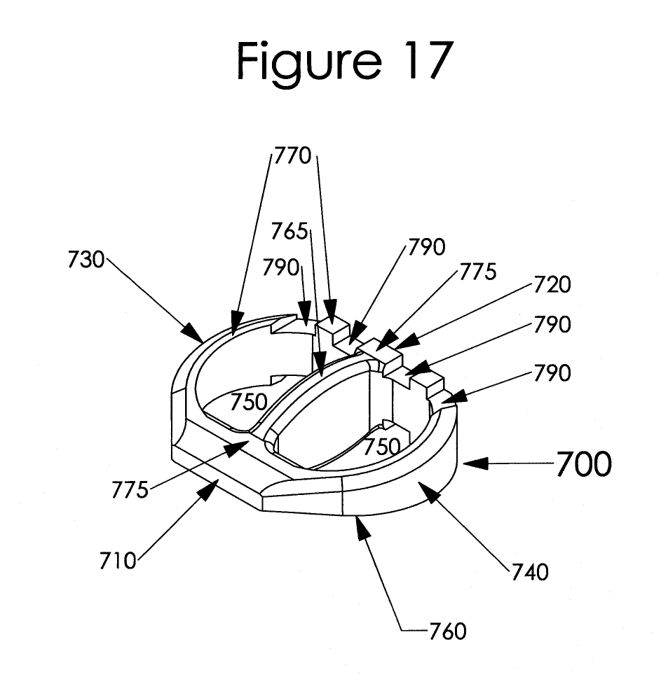

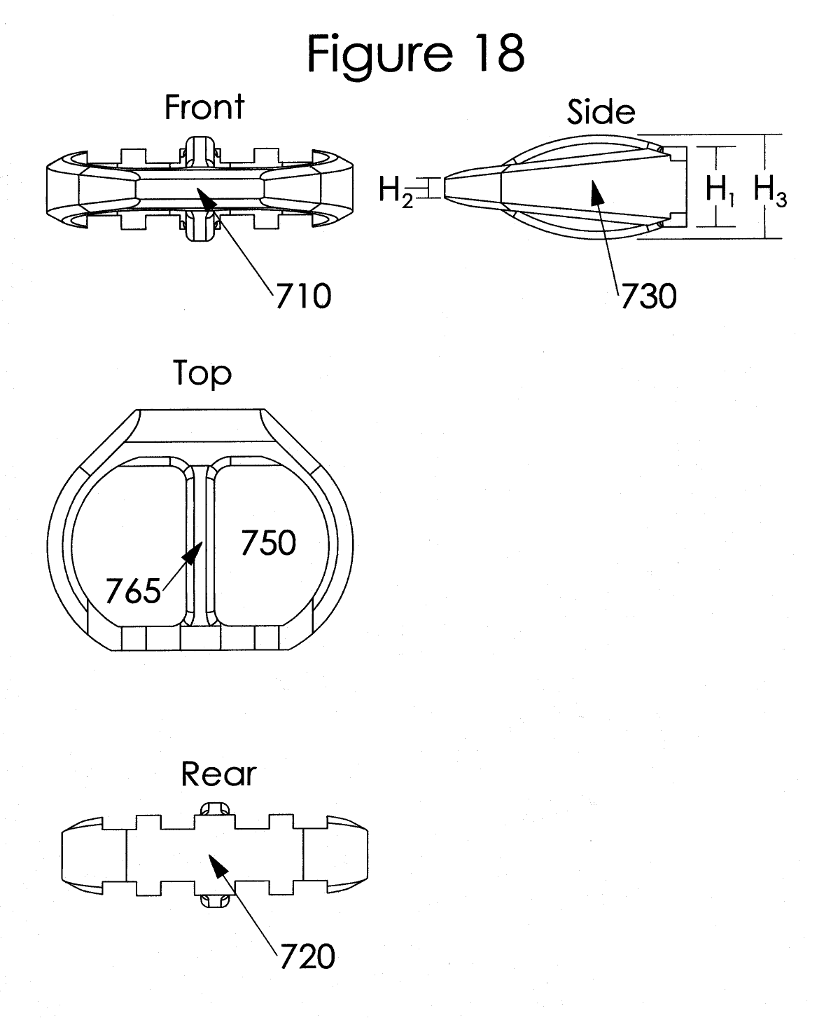

Additional embodiments of the present invention include all of the aforementioned configurations with the addition of a center member between the front end member and the back end member dividing the single aperture of those prior embodiments into two apertures. The center member can be configured about straight between the front end member and the back end member or it can be convex, concave, or irregular from a top view of the implant. Preferably, the upper surface and the lower surface of the center member are convex between the front end member and the back end member to conform to the concave configuration of the vertebral end plates. Most preferably, the maximum height of the center member is larger than the maximum height of the first side member and the second side member to create concavity for the upper surface and the lower surface between the sides of the implant. Example embodiments of the implant with the center member are shown in FIGS. 13-18. In FIGS. 13-18, center members 565, 665 and 765 have height H₃ which are larger than heights H₁ and H_{2.}

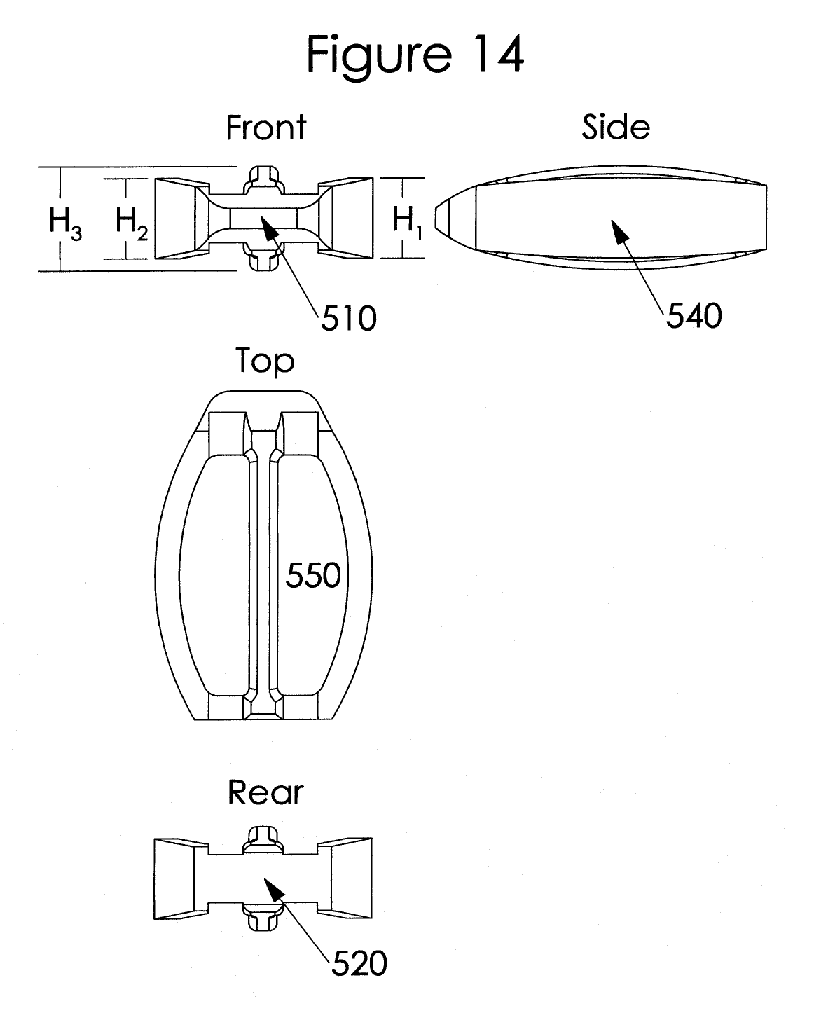

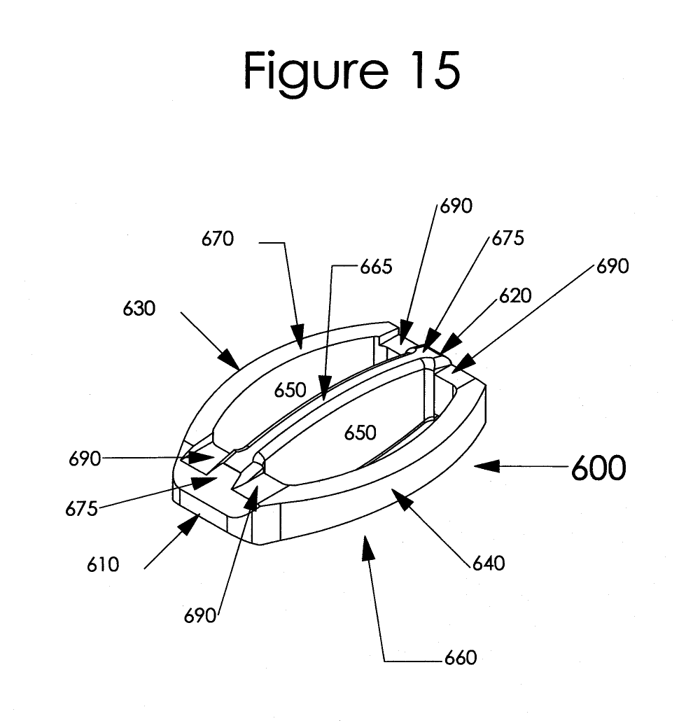

The embodiments of the invention that include the center member provide for a stronger implant and also improved maneuverability of the implant into and within the interbody disc space. Convexity in multiple directions provides for improved conformity with the end plate concave configurations, greater surface area contact with the end plates and improved support of the vertebral body end plates. As for some of the prior embodiments, the embodiments shown in FIGS. 15-18 are configured for disc spaces requiring lordosis whereas the embodiment shown in FIGS. 13 and 14 are parallel with the same heights along the lengths of the first side member 530 and the second side member 540.

In yet another embodiment of the present invention, the implant further comprises a deployable spike mechanism which remains beneath/concealed both the upper surface and the lower surface of the implant until deployment is desired. An advantage of the deployable spikes is less damage to the endplates of the vertebrae during device installation.

The deployable spike mechanism of the present invention comprises spikes within apertures in the center member that are forced out through the upper surface and/or lower surface of the implant when force is applied to the deployment mechanism. Preferably, the force is applied without impact to the implant to help avoid movement of the positioned implant. Accordingly, the present invention utilizes a turning force (torque) to minimize movement of the implant in the interbody space.

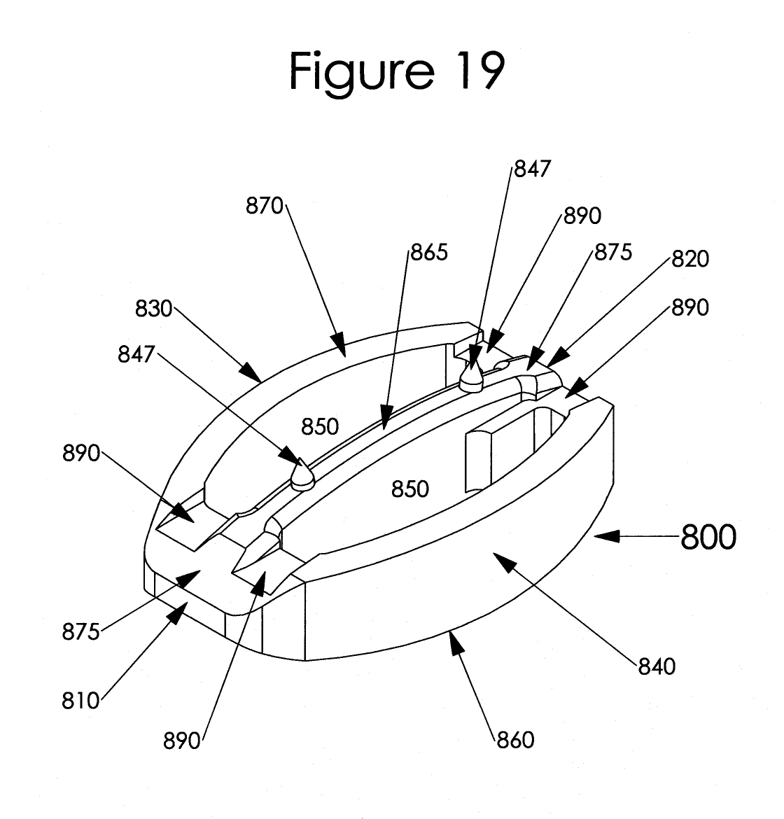

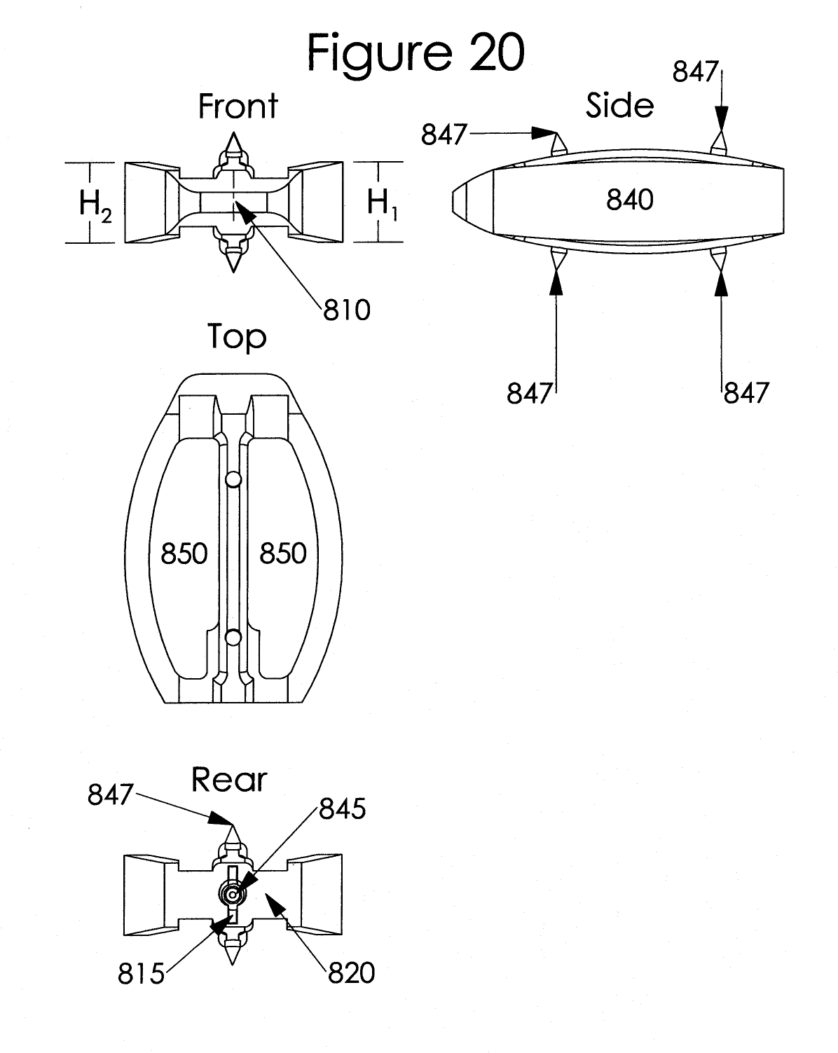

One example embodiment of the implant with a deployable spike mechanism is shown in FIGS. 19-22. The implant shown in FIGS. 19-22 is a parallel implant that is not configured for lordosis, i.e., the heights of the first side member 830 and the second side member 840 are the about the same along the lengths of them.

Implant 800 comprises front end member 810, back end member 820, first side member 830, second side member 840, center member 865 and apertures 850 generally extending through the implant from the lower surface of the implant (designated 860) to the upper surface of the implant (designated 870). Front end member 810 is ramped upwards towards the back end member 820. The outer surfaces of first side member 830 and the second side member 840 are convex and the inner surfaces are concave. It is understood that the inner surfaces of the first side member 830 and the second side member 840 may also be straight, convex or irregular configurations. Recesses 890 are formed on both of the front end member 810 and the back end member 820 between the first side member 830 and the second side member 840 on both sides of the center member 865. The recesses are in the upper surface 870 and the lower surface 860. Recesses 890 are configured for receiving and removably attaching to/fastening to an installation device (e.g. clamp) used to position and install the implant 800 in the interbody disc space. The size of the recesses 890 may be, but need not be, configured slightly larger than the connecting elements of an installation device for stability between the two. Bone graft material (not shown) may be packed within apertures 850 of implant 800 prior to installation of implant 800 in a disc space. Again, the installation device preferably includes components that cover the apertures at or at about the upper surface 870 and the lower surface 860 of the implant 800 to keep bone graft material in the implant 800. The maximum height H₁ of first side member 830 is about equal to the maximum height H₂ of second side member 840.

Preferably, the upper surface 870 and the lower surface 860 of the center member 865 are convex between the front end member 810 and the back end member 820 to conform to the concave configuration of the vertebral end plates. Most preferably, the maximum height H₃ of the center member 865 is larger than the maximum heights of the first side member 830 and the second side member 840 to create concavity for the upper surface and the lower surface between the sides of the implant as well. The upper surface 870 and the lower surface 860 of implant 800 is convex from the front end member 810 to the back end member 820 and from one side member to the other.

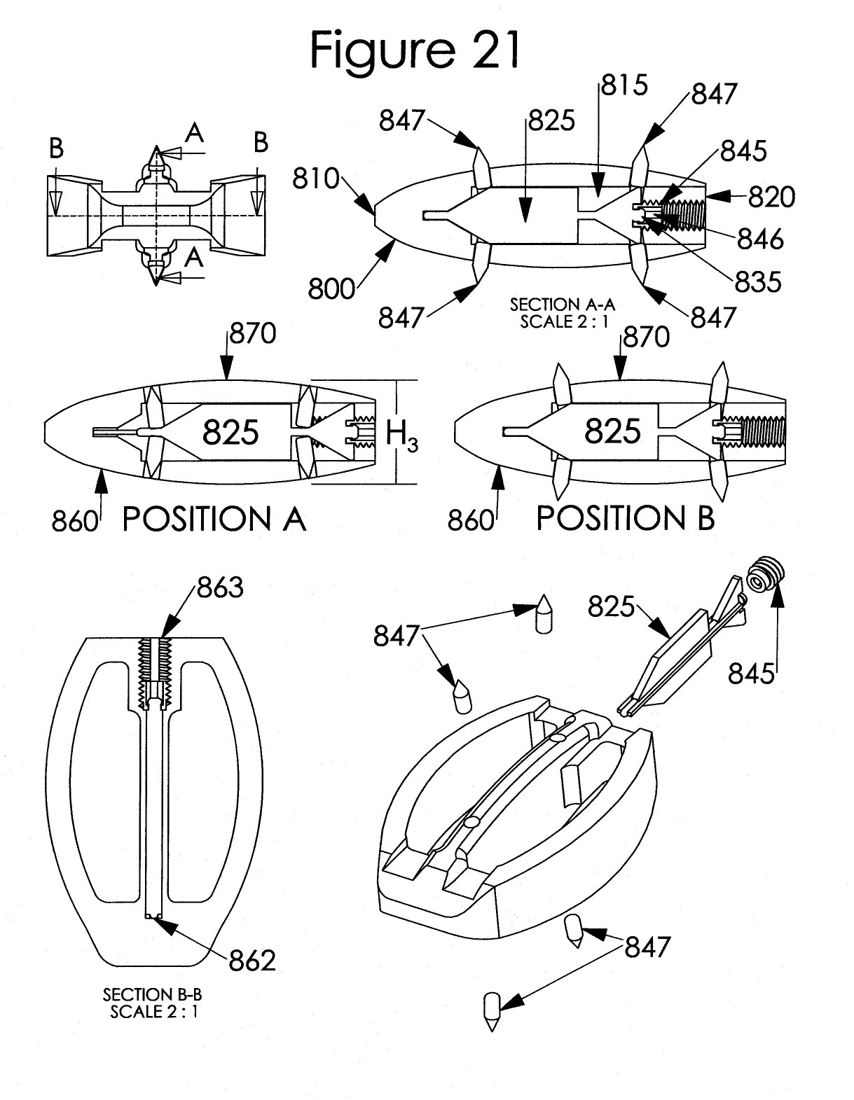

Spikes 847 are shown deployed outside the outer surfaces (860 and 870) of the implant 800. FIG. 21 shows the implant 800 with the spikes within the implant's upper surface 870 and lower surfaces 860 prior to deployment. The spike deployment mechanism is located within the members of the implant to avoid protrusions so that the implant 800 can be used and installed without deploying the spikes 847, if desired. Implant 800 includes an internal aperture 815 with a front end 862 and back end 863. The back end 863 of the internal aperture 815 comprises a threaded opening configured to receive a screw head. Internal aperture 815 extends to the upper surface 870 and the lower surface 860 of implant 800 through ports that are shown circular in the embodiment shown in FIGS. 19-22 but other shapes are possible and within the scope of the invention.

The spike deployment mechanism further comprises a wedge shaped advancement pin 825 configured to fit within the internal aperture 815. As shown in FIG. 21, when in Position A, spikes 847 are located near the lower portions of the wedge shapes on pin 825. When the pin 825 is advanced to Position B using by inserting a screw driver into aperture 846 of screw head 845 thereby advancing pin 825 and screw head 845 forward in the threaded opening of internal aperture 815, the wedged configuration forces the spikes 847 out of the center member 865 of implant 800.

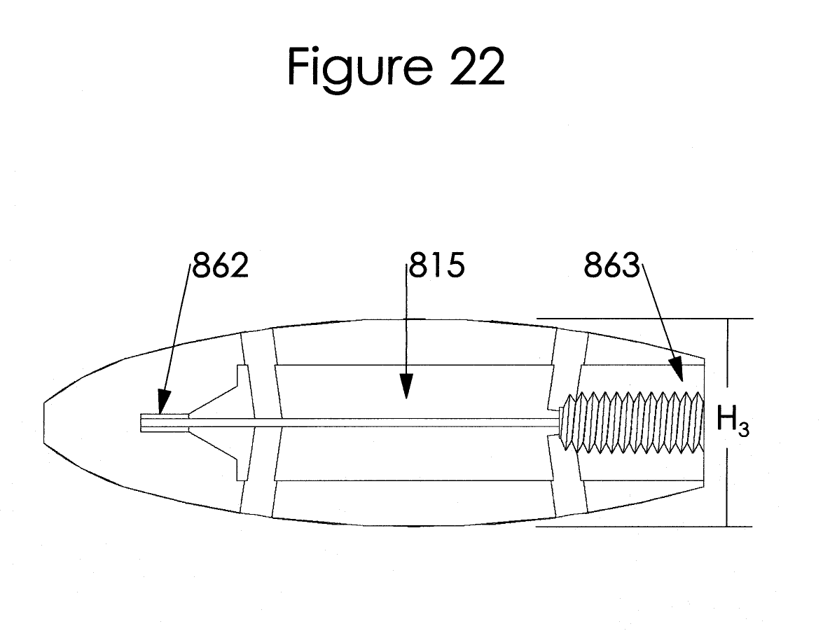

In the embodiment shown in FIGS. 19-22, the spike deployment mechanism is also retractable. The back end of pin 825 includes a lipped protrusion 835. The screw head 845 may be configured for a compression fitting at its front end for a compression fitting over lipped protrusion 835 such that the pin 825 may be pulled back towards the back end of implant 800 by reversing the turn of screw head 845. The lipped protrusion 835 being fitted within the aperture of the screw head 845 provides a means to pull the pin back towards the back end when the screw head 845 is directed out of the internal aperture 815. When the pin 845 is returned to Position A, the spikes 847 are free to slide down into internal recess 815. This function is especially useful for moving an implant after it was thought to be in place but needs to be adjusted after imaging. FIG. 22 is a cross section of the embodiment of the implant shown in FIG. 21 without the pin 825, spikes 847 and screw head 845. The aperture 815 is shown.

It is understood that the present invention includes all of the implants described herein that contain a center member without and with the spike deployment mechanism, including implants configured parallel and for lordosis.

Implant 800 is installed and positioned in the disc space with the spikes in the first position (“Position A”) until the implant is in the desired location. A screw driver (not shown) is then inserted into internal aperture 815 and into screw head 845. As the screw driver is turned, pin 825 and screw head 845 advance and spikes 847 are forced out of the center member 865 (into the endplates of the vertebral body) and into Position B.

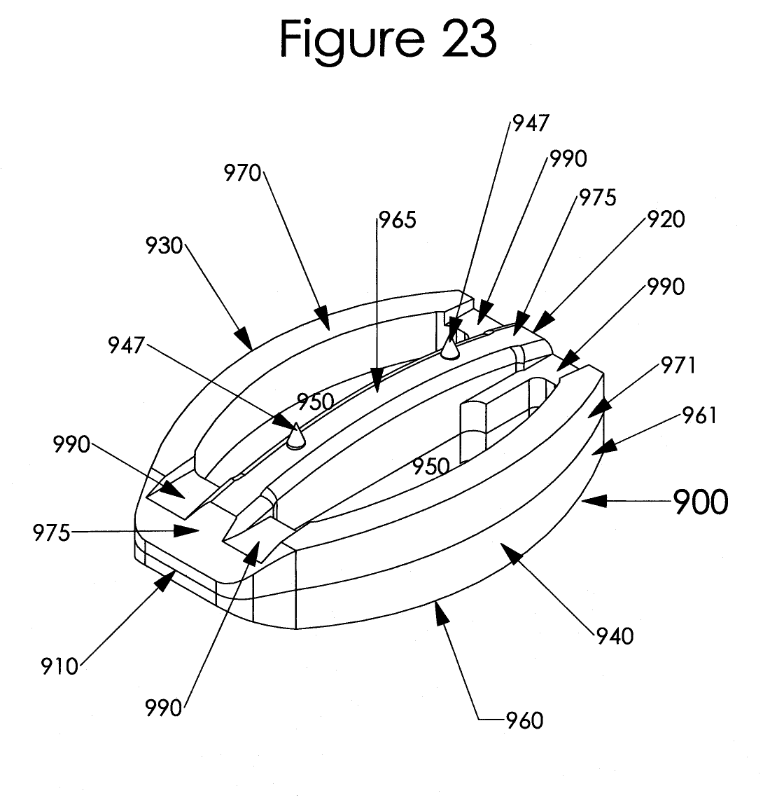

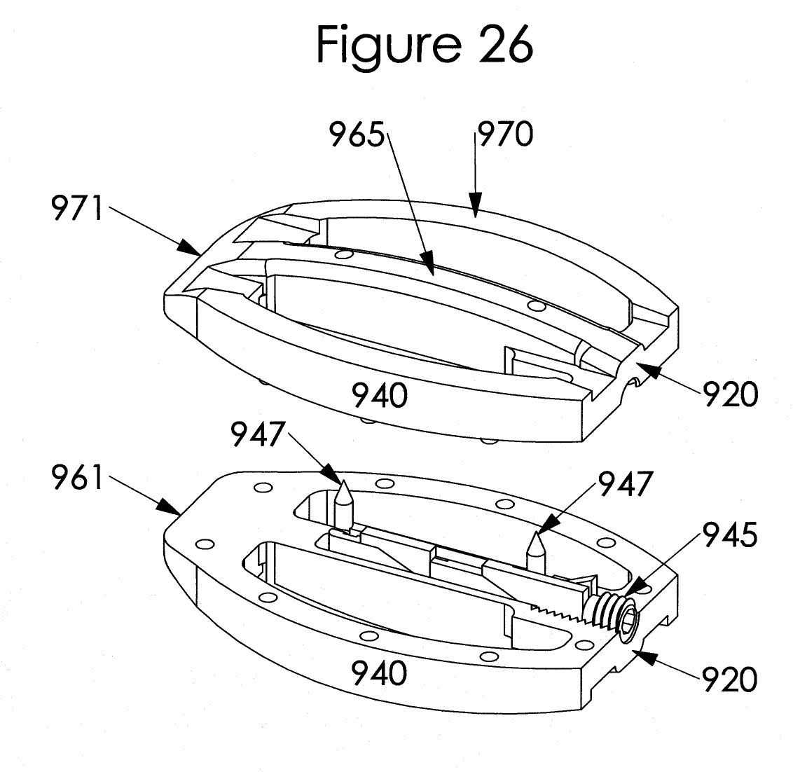

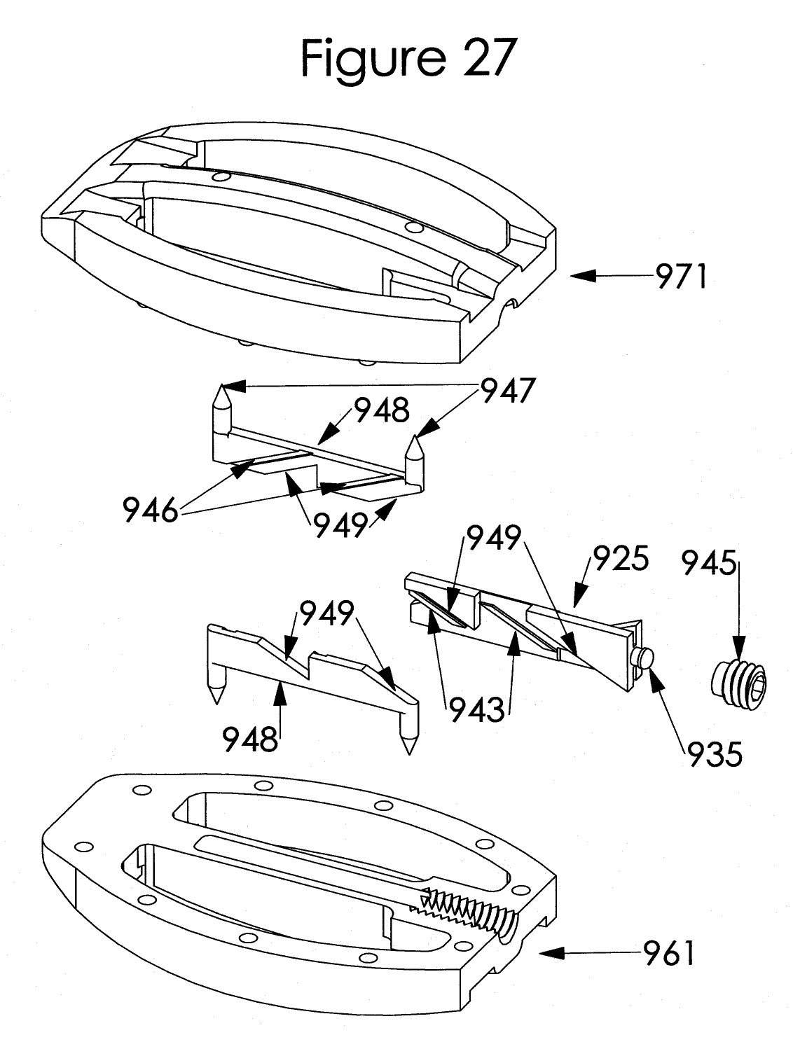

Yet another embodiment of the present invention is shown in FIGS. 23-27. The implant 900 is made from two pieces, an upper half 971 and lower half 961. Any of the implants according to the present invention can be configured in this manner. Having the implant 900 manufactured in two pieces and then secured together may assist with the manufacture of the implants of the present invention whether with or without the spike deployment mechanism shown in FIGS. 23-27. The present invention also includes the implant shown in FIGS. 23-27 manufactured as one piece instead of two as shown in FIGS. 23-27. When implants of the present invention are manufactured in halves and then fastened together, the parts can be secured together using glue, screws, or the like (not shown). Use of ridges, grooves, dimples, holes, etc. between the parts will increase the shear strength of the implant and is included in the scope of the invention.

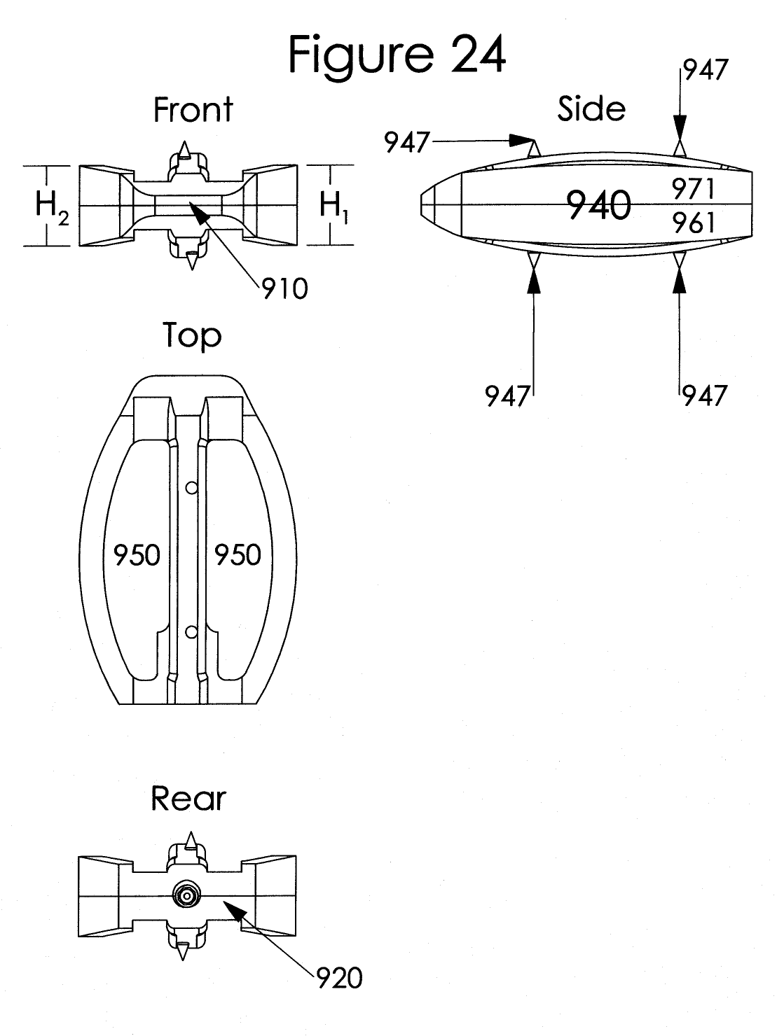

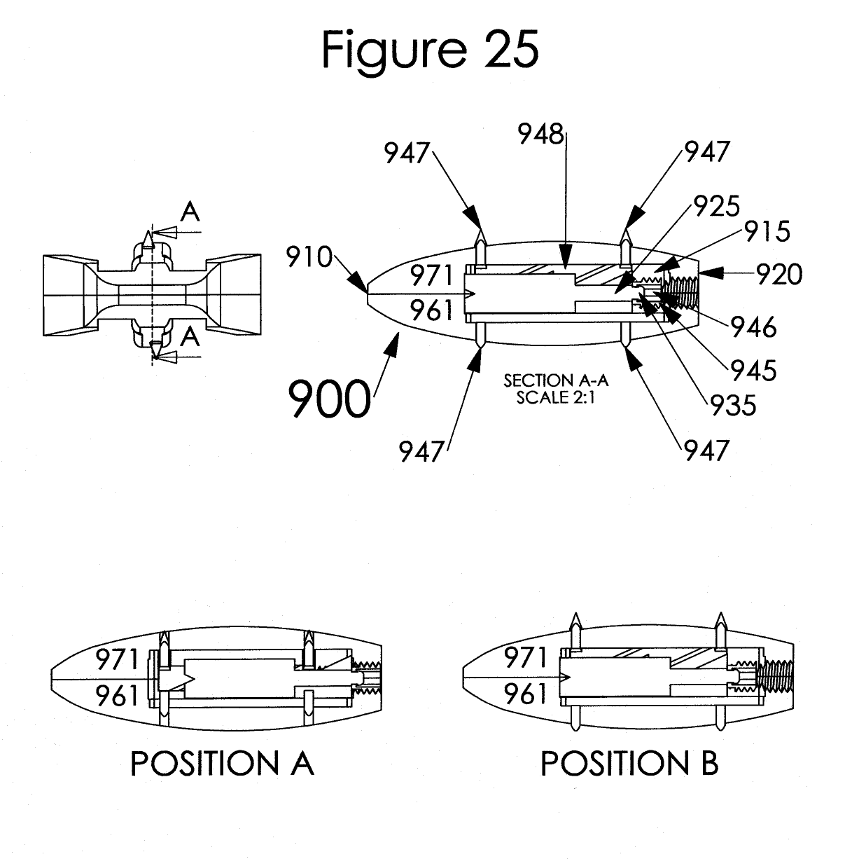

As shown in FIGS. 25-27, spikes 947 are paired on each side (upper and lower) of the implant and connected by a spike body 948. Spike bodies 948 with spikes 947 are positioned on both sides of pin 925. Accordingly, in this embodiment, as also shown in FIGS. 24 and 25, the spikes are slightly offset from the middle of the center member 965 on the upper surface 970 and the lower surfaces 960. Spike bodies 948 and pin 925 include angled surfaces 949 that move when pin 925 is moved. Spike bodies 948 also include channels/grooves configured to receive the raised bars 943 on the pin 925. The bar 943 and groove 946 configuration functions to help raise/deploy the spikes 947 and to also lower/retract the spikes 947 when the pin is moved either directly by the screw driver (not shown) and/or when the screw head 945 is turned.

When the pin 925 is advanced from position A to Position B, the angled/wedged configurations and the channels/bars forces the spike bodies 948 out of the center member 965 of implant 900 thereby deploying the spikes 947. Turning the screw head 945 in the opposite direction retracts the spikes 947 by pulling the spike bodies 948 into the implant 900 due to the angled channel and bar configuration.

It is understood that the present invention is not limited to implants with only two spikes on the upper side and the lower side of the implant and that implants with other numbers of spikes (e.g., one, three or four on top and/or on bottom) are included in the scope of the invention.

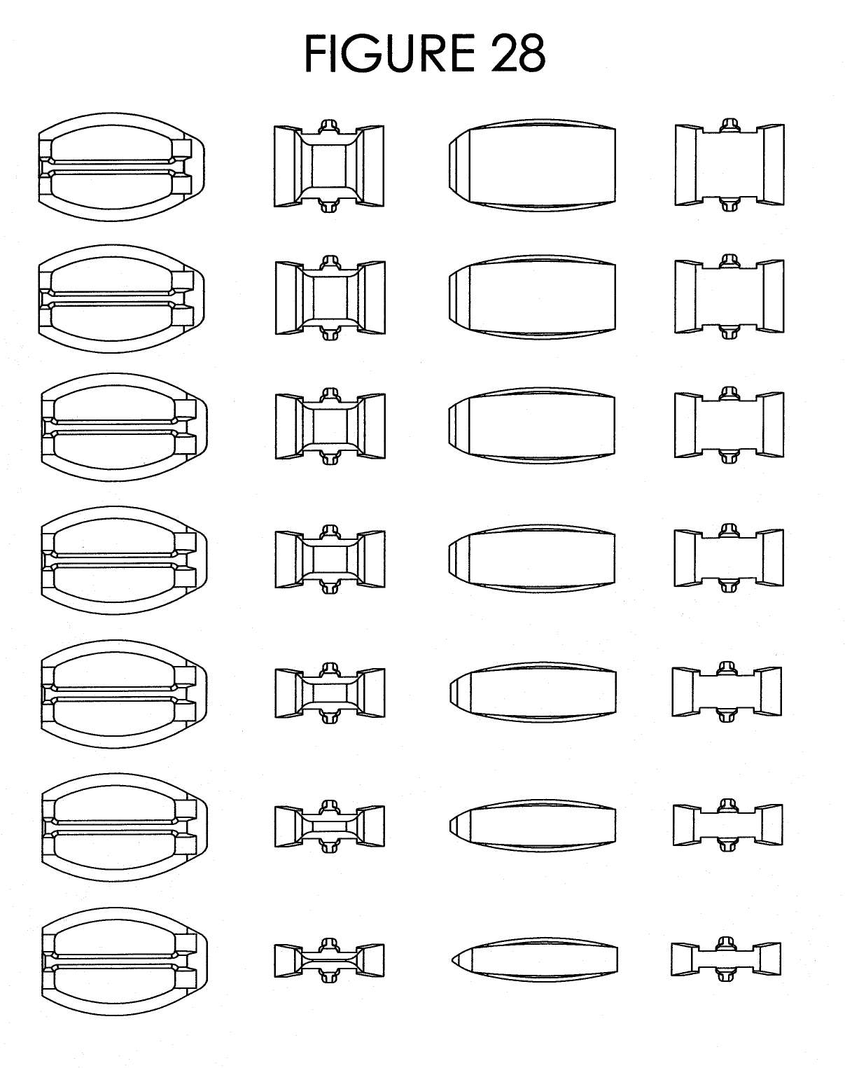

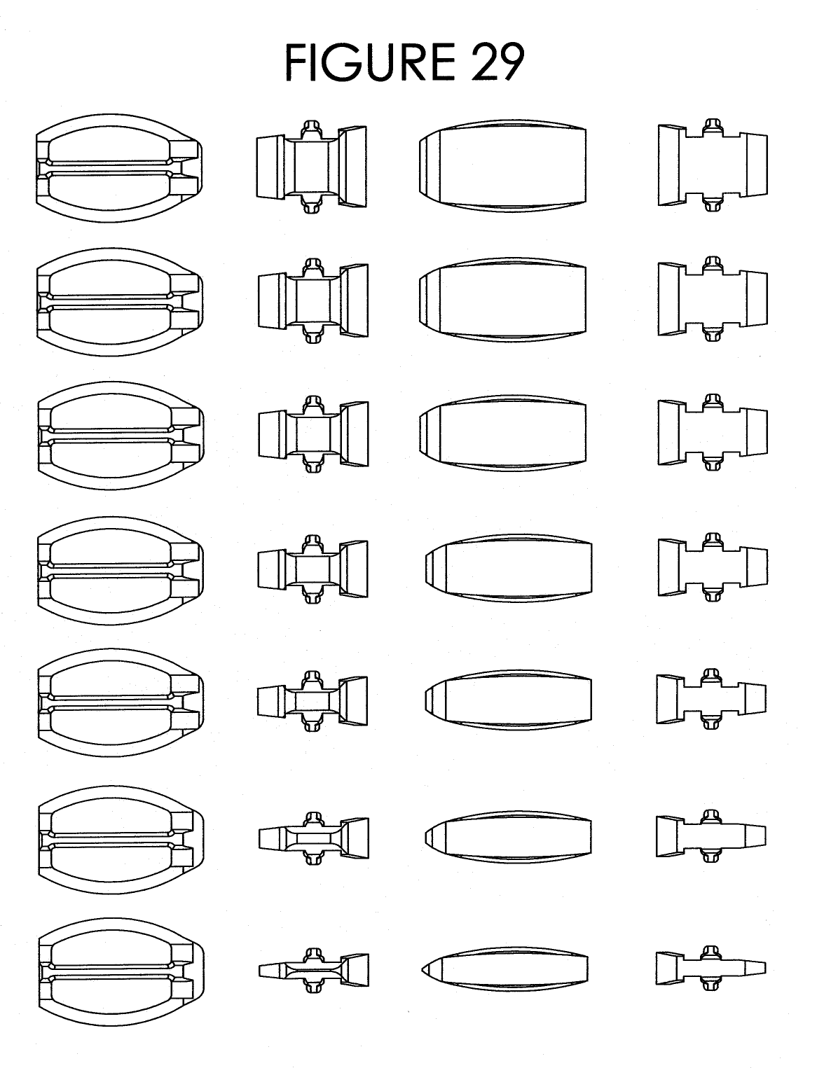

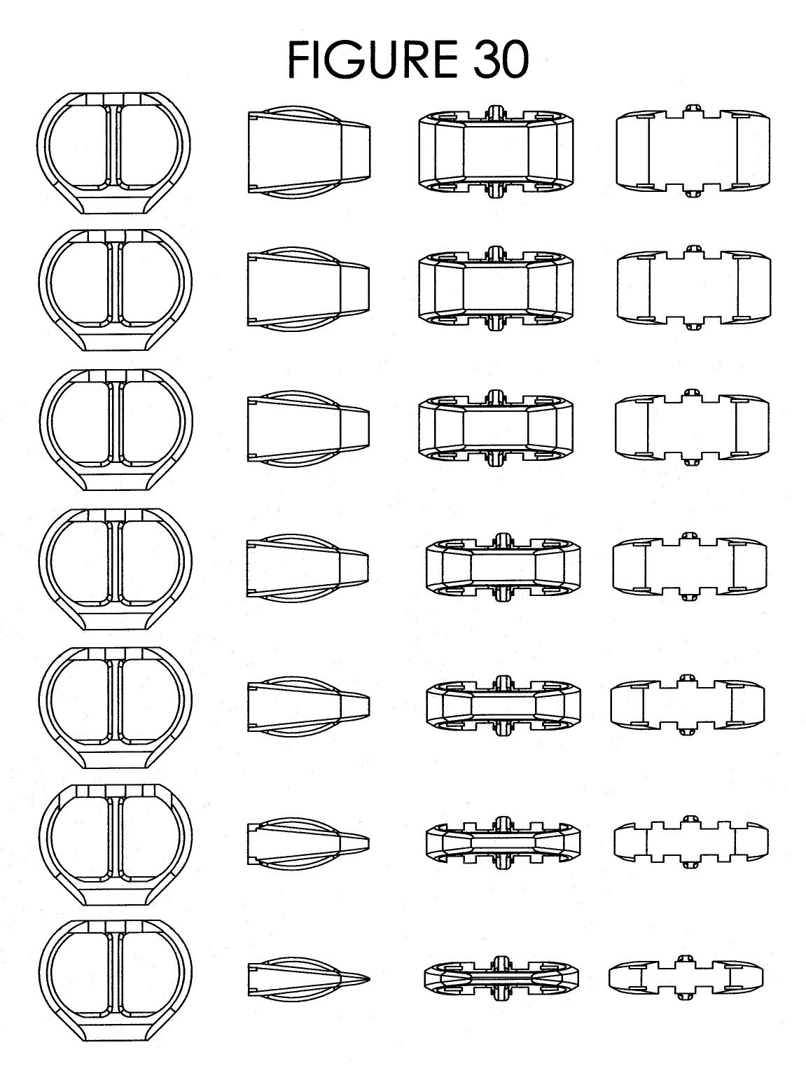

FIGS. 28, 29, 30 show several embodiments of the implant of the present invention in various sizes and configurations.

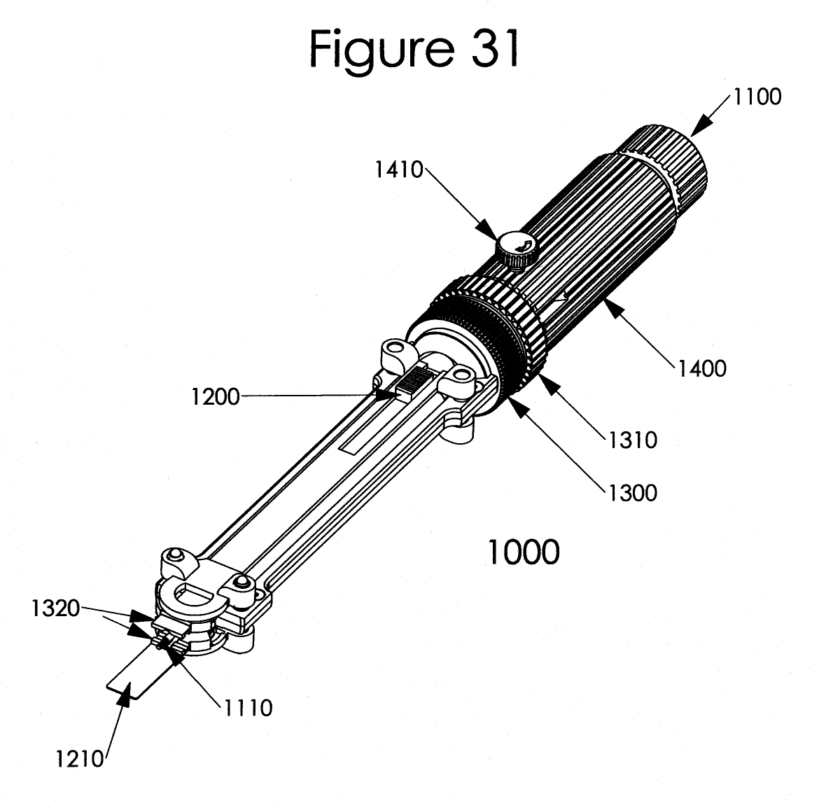

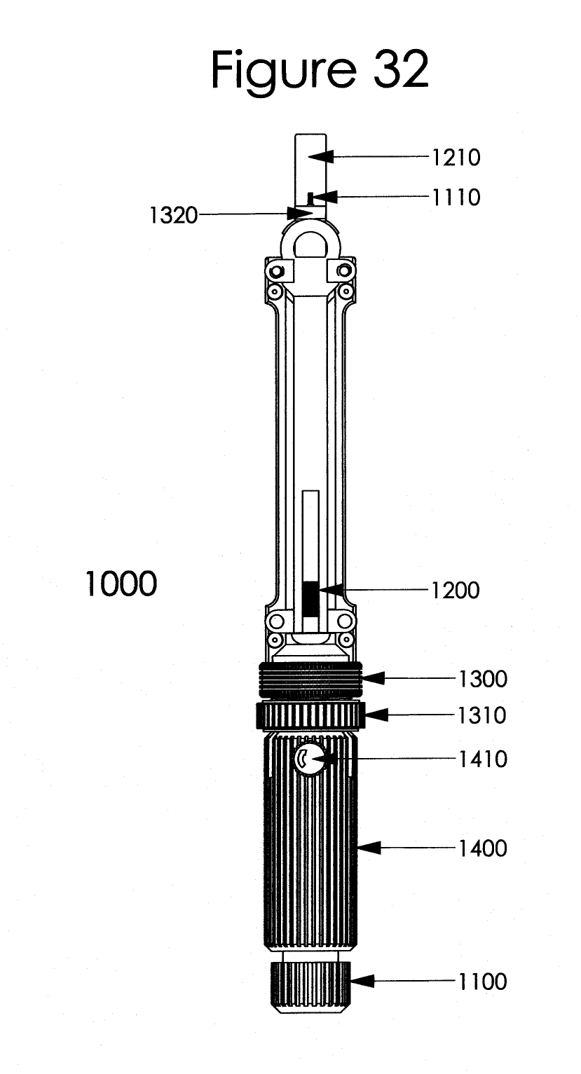

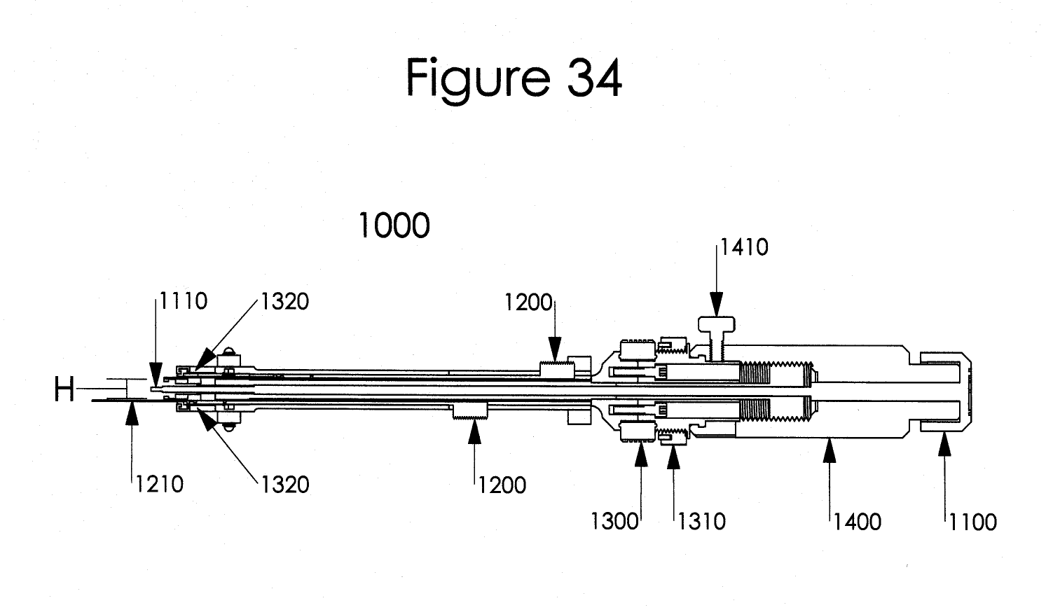

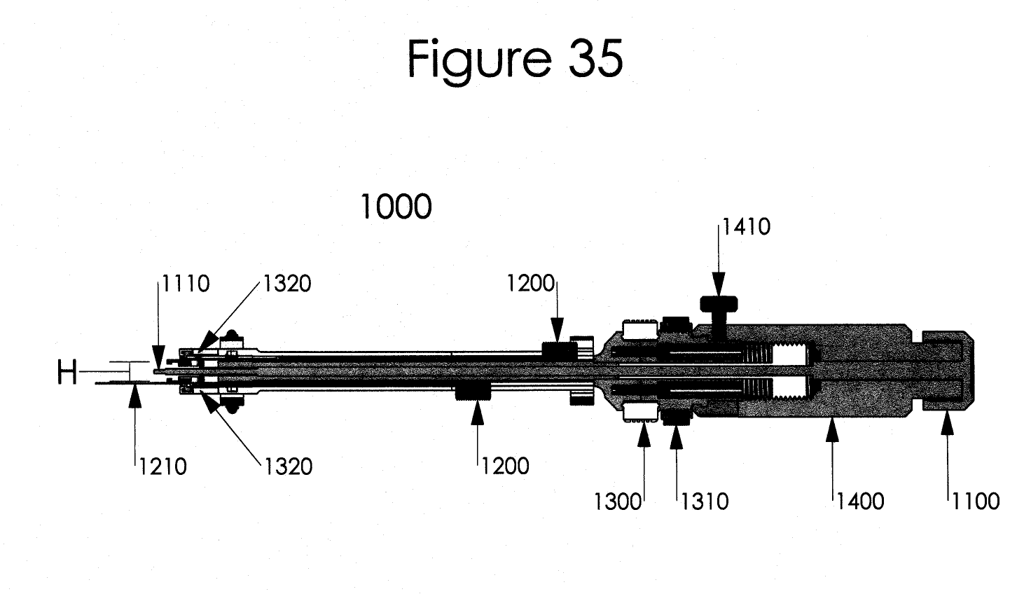

The present invention also includes the novel installation device for the implants. One embodiment of the installation device capable of removably clamping to the back end member of the implant with independent sliding covers on top and bottom for the implant aperture(s) is shown in FIGS. 31-38. The installation device can be used as an impactor and it also includes an opening down the axis of the device for insertion of a screw driver.

Device 1000 includes screw driver 1100 having screw head 1110, sliding covers 1210 connected to sliding tab 1200, turning knob 1300 to raise and lower the clamps 1320 with locking knob 1310 and center impactor knob 1400 to advance and retract the center impactor against a clamped implant.

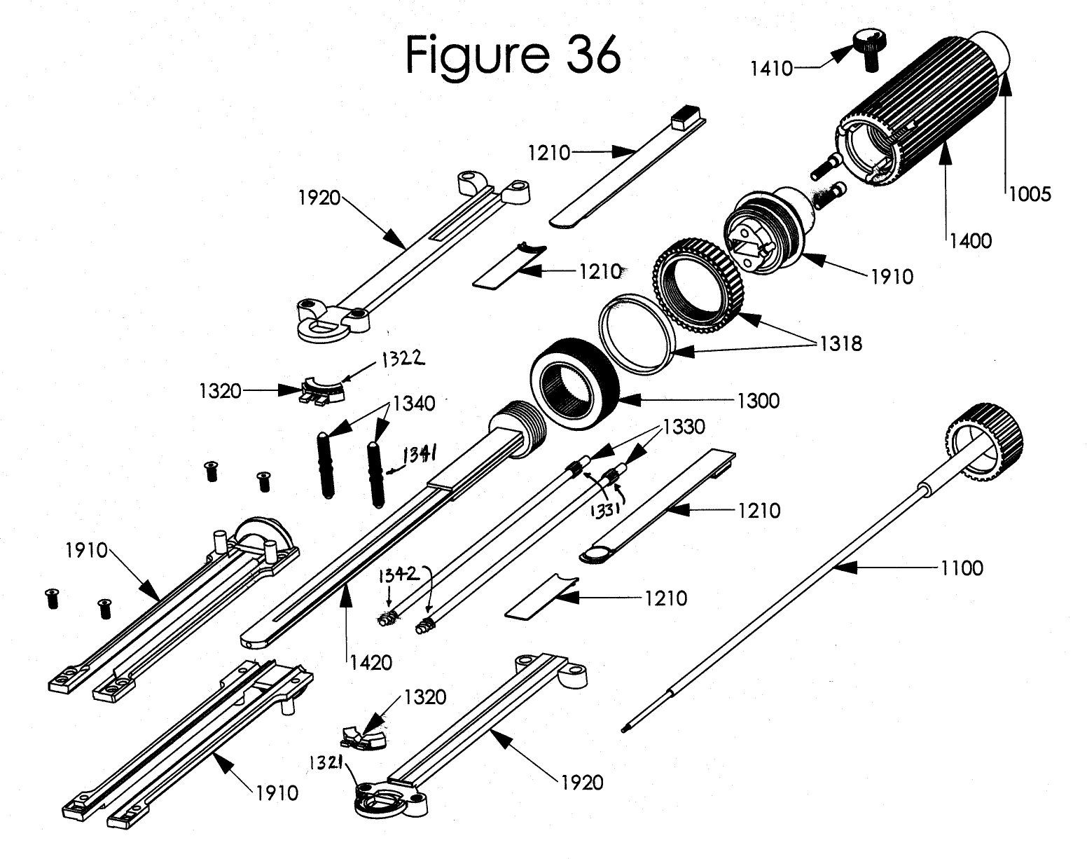

FIG. 36 is an exploded perspective view of the embodiment showing the mechanical parts and inner workings of the device.

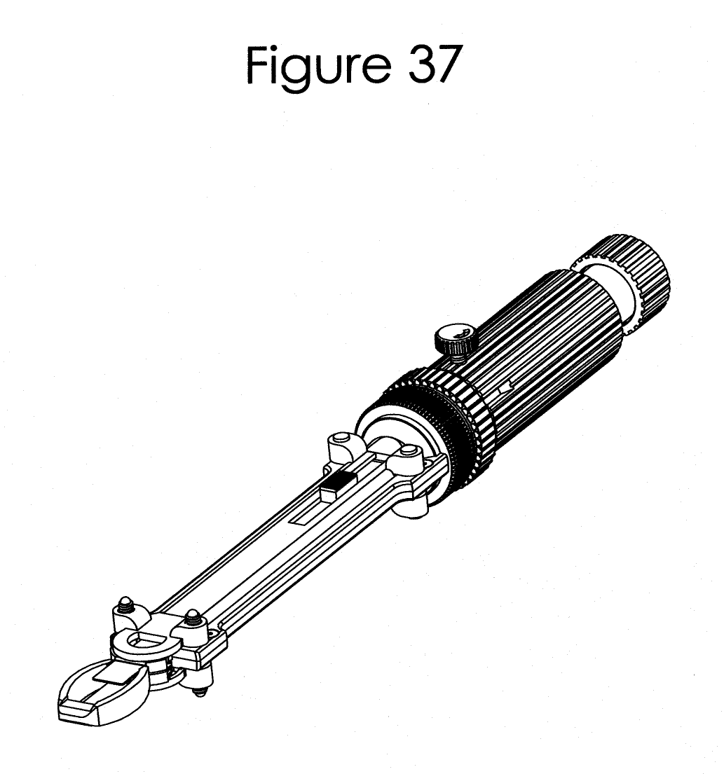

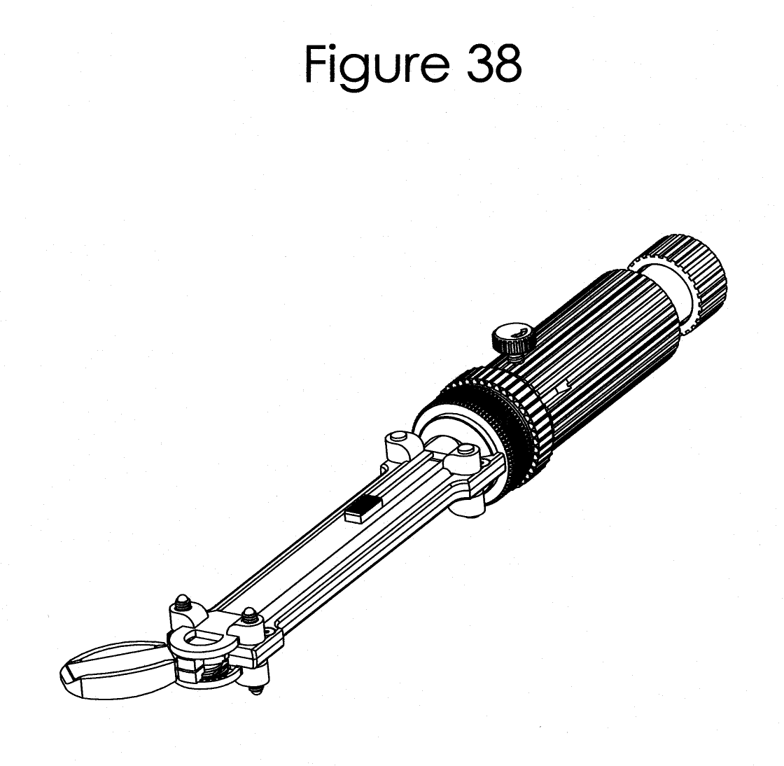

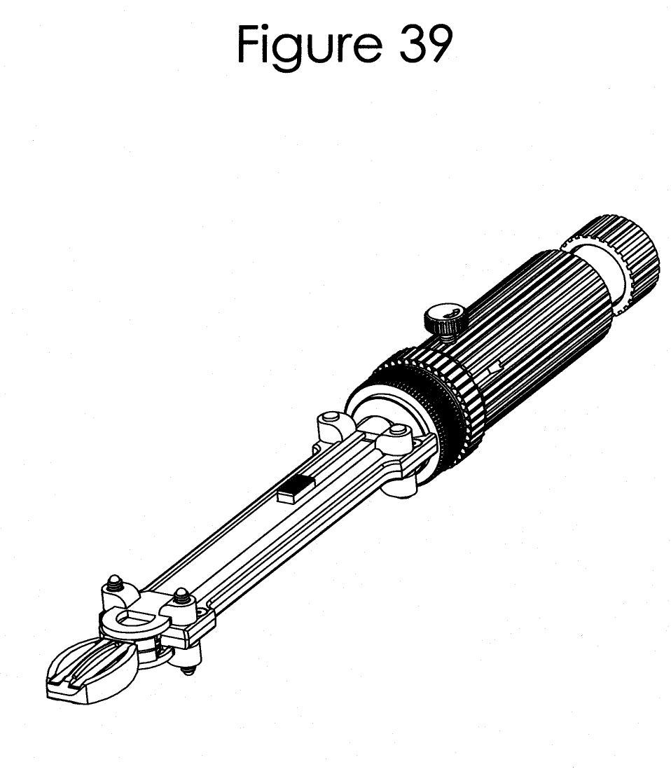

FIGS. 37-38 are perspective views of the installation device removably secured to an implant of the present invention. The installation device in FIGS. 37 and 38 are for implants without a center member. FIG. 38 shows the implant attached to the installation device with the implant rotated about the tip of the installation device. This feature is particularly useful during installation when obstructions prevent direct access to the disc space. For the embodiment of the invention shown in FIG. 39, the covers of the installation device are shown with a split configuration to accommodate an implant with a center member.

Use and the components of the installation device 1000 for installation of a bone graft packed implant are now described. In the embodiments for an installation device shown in FIGS. 31-39, the installation device, with the screw driver 1100 removed, is clamped on an implant using the clamping feature (clamps 1320) of the device using geared shafts 1330, rounded screws 1340, geared knob 1300, and clamping locking mechanism 1310. When center impaction is desired, the center impactor 1420 is advanced putting the center impactor 1420 in direct contact with the back end of the implant. The locking screw 1410 is secured to lock the center impactor in place. The lower cover 1210 is then extended over the lower surface of the implant. The implant is then packed with bone graft material. The upper cover 1210 is then extended over the upper surface of the implant. The installation device 1000 and implant are then inserted into the patient with the ramped front end of the implant in front. The implant is placed into the disc space. If needed or desired, the back end of the installation device 1005 can be impacted to separate the endplate of the vertebral bodies and force the implant into the desired position. Once located, for implants with spikes, the screw driver 1100 is inserted down the installation device and turned to deploy the spikes. After confirming the installation location with x-rays, the covers 1210 are removed by sliding them back, the clamp is unlocked and opened and the installation device 1000 is removed from the patient. Housing parts 1910 and 1920 and associated hardware (screws) complete the installation device.

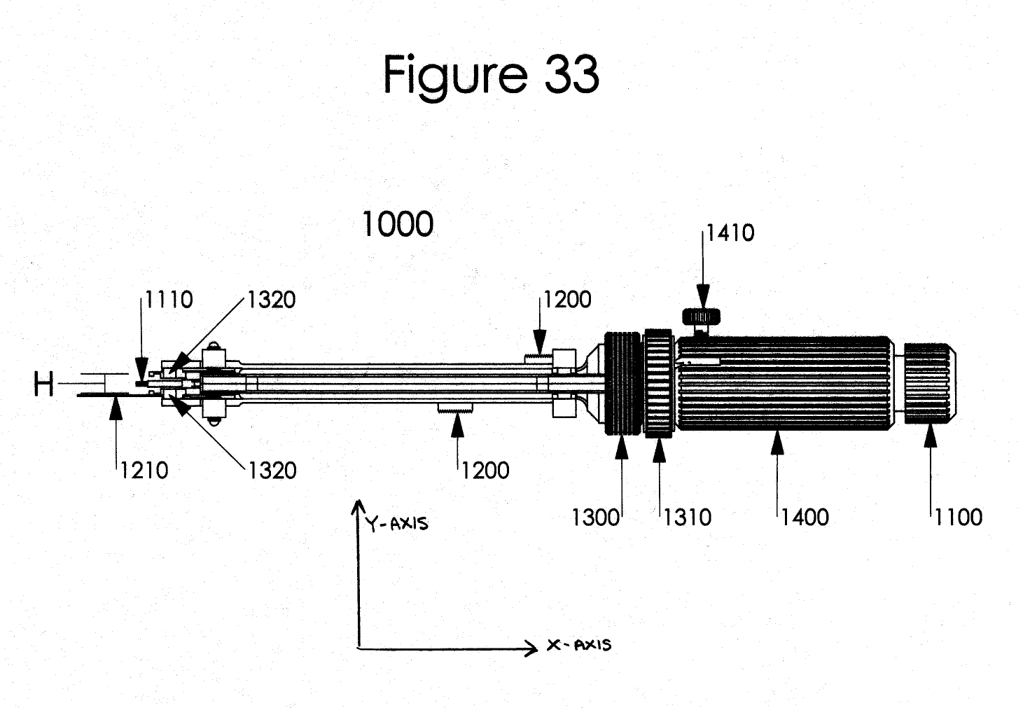

Accordingly, the present invention includes a spinal implant installation device comprising an impactor 1420 having a first end (near clamp 1320) and a second end (located near screw driver 1100) located between a top housing (shown as 1910 and 1920 above the impactor 1420 in FIG. 36) and a bottom housing (shown as 1910 and 1920 below the impactor 1420 in FIG. 36) each of the top housing and the bottom housing having a first end and a second end. The first end of said impactor 1420 is capable of sliding out past said first ends of said top housing and said bottom housing when handle 1400 is rotated. The top housing and the bottom housing are connected to each other by rounded screws/worm gears (for safety inside the patient) capable of increasing and decreasing the distance between the housings when the screws 1340 are rotated. The center portions of the screws 1340 are straight geared 1341 to engage the adjacent worm gears 1342 on shafts 1330. The implant is inserted into the patient in the X-axis direction (see FIG. 33) and the clamp opens and closes along the Y-axis, perpendicular to the axis of the installation device. Screws 1330 have gears (1331 and 1431) in the center portions of each of them that engage the worm gears 1342 on the geared shafts 1330 which in turn have gears at the other end of the shafts 1330 near the geared knob 1300.

The clamps 1320 of the device are removably attached to the top and bottom housings. The clamps 1320 are configured to attach to an implant when the implant is positioned between the clamps 1320 and clamps 1320 are closed. The spinal implant installation device of the present invention also includes clamps 1320 that can rotate about an axis while the implant is positioned between them. The clamps comprise arced protrusions 1322 compatible with arced grooves 1321 on the first ends of the housings capable of rotating each of the clamps about an axis perpendicular to the X-axis of the device and near the first end of the device. In a preferred embodiment, the clamps also include notches and ridges along the protrusions 1322 and grooves 1321 to help secure the clamp in an angled position when the clamp is rotated.

The top housing and the bottom housing comprise (but the device could also be made and/or used with only one) an internal aperture between the first end and the second end of the housing. The implant covers 1210 are positioned inside the internal aperture of the housings 1920 and are capable of sliding along the axis of the device (the Y-axis) inside the housings and out past the first ends of the housings to cover apertures in an implant.

In another embodiment of the invention, an implant and an installation device are used together as an intervertebral implant system. For example, using any of the aforesaid embodiments of the implant in combination with an installation, the resulting intervertebral implant system can be used for installation of an intervertebral implant.

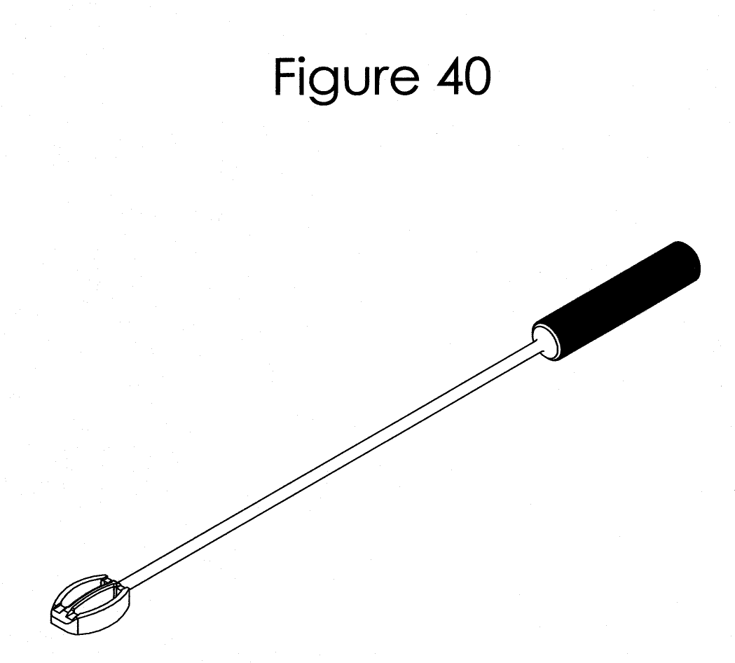

During a surgery, the implant size is determined using test implants, typically made of metal, which are placed in the intervertebral space after discectomy. Utilizing a trial and error approach, a surgeon will place metal implants of varying sizes. The primary difference between the sizing implants and the final implant is that the final implant is typically made of a biocompatible material. Keeping sizing implant the same as the actual implants decrease the chance that the implant will be improperly sized. The present invention also includes an improved sizing device as shown by way of example for one embodiment of the implant invention in FIG. 40 which is a removably attached handle to an implant of the present invention. The handle can be clamped to the implant or it can be threaded into the internal aperture of the implant. It being understood that any one of the implant configurations in the present invention can be used with clamps or handles for sizing devices.

I claim:

1. An interbody spinal implant device comprising:
a ramped front end member and a flat outer surfaced back end member both fixedly attached to a first side member and a second side member;
convex outer surfaces on said first side member and said second side member and straight inner surfaces on said first side member and said second side member;
an aperture is formed within said implant between said members;
a convex upper surface on said first side member and a convex upper surface on said second side member; and
a convex lower surface on said first side member and a convex lower surface on said second side member.

2. The implant of claim 1 further comprising a recess on at least one of said upper surface and lower surface of said back end member configured to removably attach to an installation device.

3. The implant of claim 2 further comprising a recess on at least one of said upper surface and lower surface of said front end member configured to removably attach to an installation device.

4. The implant of claim 3 wherein the maximum height between the upper surface and lower surface of said first side member is greater than the maximum height between the upper surface and the lower surface of said second side member.

5. The implant of claim 1 further comprising a recess on at least one of said upper surface and lower surface of said front end member configured to removably attach to an installation device.

6. An interbody spinal implant device comprising:
a ramped front end member and a flat outer surfaced back end member both fixedly attached to a first side member, a second side member and a center member;
convex outer surfaces on said first side member and said second side member and concave inner surfaces on said first side member and said second side member;
two apertures between the front end member and the back end member within said implant, the first aperture between said first side member and said center member and the second aperture formed between said second side member and said center member;
a convex upper surface on said first side member, a convex upper surface on said second side member, and a convex upper surface on said center member; and
wherein the maximum height between the upper surface and lower surface of said center member is greater than both maximum heights between the upper surface and lower surface for said first side member and said second side member.

7. The implant of claim 6 further comprising a convex lower surface on said first side member, a convex lower surface on said second side member, and a convex lower surface on said center member;

8. The implant of claim 7 further comprising a recess on at least one of said upper surface and lower surface of said back end member configured to removably attach to an installation device.

9. The implant of claim 8 further comprising a recess on at least one of said upper surface and lower surface of said front end member configured to removably attach to an installation device.

10. The implant of claim 9 further comprising an internal aperture within said center member and through said back end member with a threaded opening at the distal end of said internal aperture comprising therein a deployable and retractable spike mechanism for deploying at least two spikes, said spike deployment mechanism comprising a wedge shaped pin removably attached to a screw head and at least two spikes, wherein said spikes are forced out of the outer surface of said center member when said screw head is rotated and retract back into said center member when said screw is rotated in the opposite direction.

11. The implant of claim 10 wherein two spikes are fixedly connected to each other by a spike body, wherein said spike body comprises angled surfaces and grooves compatible with angled surfaces and raised bars on said pin.

12. The implant of claim 10 wherein two sets of spikes are each fixedly connected to each other by two spike bodies, wherein said spike bodies each comprise angled surfaces and grooves compatible with angled surfaces and raised bars on opposite sides of said pin.

13. The implant of claim 12 wherein the two sets of spikes each deploy and retract through opposite surfaces of said implant.

14. The implant of claim 9 wherein the maximum height between the upper surface and lower surface of said first side member is greater than the maximum height between the upper surface and the lower surface of said second side member.

15. The implant of claim 7 further comprising a recess on at least one of said upper surface and lower surface of said front end member configured to removably attach to an installation device.

16. An interbody spinal implant system comprising:
an implant device comprising a ramped front end member and a flat outer surfaced back end member both fixedly attached to a first side member and a second side member, convex outer surfaces on said first side member and said second side member and straight inner surfaces on said first side member and said second side member, an aperture formed within said implant between said members, a convex upper surface on said first side member and a convex upper surface on said second side member, a convex lower surface on said first side member and a convex lower surface on said second side member; and
an installation device comprising a pivoting handle opposite a clamp having two sides, a top and a bottom, each side of said clamp configured to removably attach to said implant on said first end and said back end covering said aperture on said upper surface and said lower surface of said implant.

17. The interbody spinal implant system of claim 16 wherein said aperture is completely enclosed by said sides of said clamp when said clamp is attached to said implant.

18. The interbody spinal implant system of claim 16 wherein said implant further comprises recesses on said upper surface and said lower surface of said front member and said back end member configured to removably attach to an installation device and wherein said sides of said clamp removably attach to said recesses on said implant.

19. The interbody spinal implant system of claim 18 wherein said aperture is completely enclosed by said sides of said clamp when said clamp is attached to said implant.

20. An interbody spinal implant device comprising:
a ramped front end member and a flat outer surfaced back end member both fixedly attached to a first side member, a second side member and a center member, convex outer surfaces on said first side member and said second side member and concave inner surfaces on said first side member and said second side member;
two apertures between the front end member and the back end member within said implant, the first aperture between said first side member and said center member and the second aperture formed between said second side member and said center member;
a ramping upper surface and lower surface on said first side member increasing in height from the front member to the back end member;
a convex upper surface and a convex lower surface on said center member; and
wherein the maximum height between the upper surface and lower surface of said center member is greater than both maximum heights between the upper surface and lower surface for said first side member and said second side member.

21. The implant of claim 20 further comprising a recess on at least one of said upper surface and lower surface of said back end member configured to removably attach to an installation device.

22. The implant of claim 21 further comprising a recess on at least one of said upper surface and lower surface of said front end member configured to removably attach to an installation device.

23. The implant of claim 22 further comprising an internal aperture within said center member and through said back end member with a threaded opening at the distal end of said internal aperture comprising therein a deployable and retractable spike mechanism for deploying at least two spikes, said spike deployment mechanism comprising a wedge shaped pin removably attached to a screw head and at least two spikes, wherein said spikes are forced out of the outer surface of said center member when said screw head is rotated and retract back into said center member when said screw is rotated in the opposite direction.

24. The implant of claim 23 wherein two spikes are fixedly connect to each other by a spike body, wherein said spike body comprises angled surfaces and grooves compatible with angled surfaces and raised bars on said pin.

25. The implant of claim 23 wherein two sets of spikes are each fixedly connected to each other by two spike bodies, wherein said spike bodies each comprise angled surfaces and grooves compatible with angled surfaces and raised bars on opposite sides of said pin.

26. The implant of claim 25 wherein the two sets of spikes deploy and retract through opposite surfaces of said implant.

27. A spinal implant installation device comprising:
an impactor having a first end and a second end located between a top housing and a bottom housing each said top housing and bottom housing having a first end and a second end, wherein said first end of said impactor is capable of sliding out past said first ends of said top housing and said bottom housing, said top housing and said bottom housing connected by geared screws capable of opening and closing said housings when said geared screws are rotated;
said top housing and said bottom housing each comprising an internal aperture between said first end and said second end, wherein said first end of said top housing and said first end of bottom housing each comprise clamps configured to removably attach to an implant when the implant is positioned between said clamps and said top housing and said bottom housing are closed together; and
implant covers in each said top housing and said bottom housing capable of sliding out past said first ends of said housings and configured to cover apertures in an implant.

28. The spinal implant installation device of claim 27, further comprising an aperture down the center axis of the device configured to receive a screwdriver.

29. The spinal implant installation device of claim 27, wherein said clamps comprise arced protrusions compatible with arced grooves on said first ends of said housings capable of rotating each of said clamps about said first end of said housing along the arches.

30. The spinal implant installation device of claim 27, wherein each of said covers are two pieces comprising an arced protrusion on one piece and an arced groove on the other capable of rotating a part of each of said clamps about an axis perpendicular to the axis of said device.
